(19) United States
(12) Patent Application Publication
(10) Pub. No.: US 2024/0094217 A1
(43) Pub. Date: Mar. 21, 2024

(54) METHODS OF TREATING SARS-COV-2 INFECTIONS

(71) Applicants: Qatar Foundation for Education, Science and Community Development, Doha (QA); HAMAD MEDICAL CORPORATION, Doha (QA)

(72) Inventors: Fares H. AL-EJEH, Doha (QA); Vidya MOHAMED-ALI, Doha (QA); Maryam A. Y. AL-NESF, Doha (QA)

(21) Appl. No.: 18/038,665

(22) PCT Filed: Nov. 25, 2021
(86) PCT No.: PCT/QA2021/050024

§ 371 Date: May 24, 2023

Related U.S. Application Data
(60) Provisional application No. 63/118,459, filed on Nov. 25, 2020.

(51) Int. Cl. G01N 33/68; A61P 31/14

(52) U.S. Cl. CPC G01N 33/6842; A61P 31/14; G01N 2800/26

(57) ABSTRACT

The present disclosure, in part, relates to novel, relates to novel, prognostic tools for severe COVID-19 disease, including identification of a set of clinical parameters that can be used to generate a clinical risk score of COVID-19 complications. The present disclosure also relates to proteomic panel-profiling of patients with severe COVID-19 complications versus mild-moderate symptoms to characterize biological processes and pathways associated with disease severity. Also disclosed are identified molecular changes associated with the clinical findings that can be used to generate a molecular severity score. In addition, the methods disclosed here relate to identifying effective methods of treatments based on the patient analysis.

### RELATED APPLICATIONS

This application claims priority of U.S. Provisional Patent Application No. 63/118,459, filed Nov. 25, 2020, the entire content of which is incorporated herein by reference.

### BACKGROUND

The rapid and wide-spread dissemination of the severe acute respiratory syndrome coronavirus 2 (SARS-CoV-2) has pressured, and tested, healthcare systems globally. To date, there have been over 254 million individuals infected worldwide, leading to over 5 million deaths due to the Coronavirus disease 2019 (COVID-19).

The International Severe Acute Respiratory and Emerging Infections Consortium (ISARIC) released its latest comprehensive report on 14 Jul. 2021 including data from 30 Jan. 2020 to 25 May 2021 for 442,643 individuals with laboratory-confirmed SARS-CoV-2 infections from more than 1,600 sites across 61 countries. Patients were split equally between males (221,591) and females (220,390), with a median age of 60 years. The most common comorbidities at admission were hypertension (41%), smoking (35%), diabetes mellitus (28%), cardiovascular disease (17%), and obesity (12%)1. The five most common symptoms at admission were shortness of breath, cough, history of fever, fatigue, and altered consciousness or confusion. Oxygen saturation (SpO2%) less than 94% was present in 34.8% and 25.3% of the patients who were and were not on oxygen therapy at admission, respectively. Admission to intensive care or high dependency units (ICU/HDU) at some point of illness, which could be defined as severe COVID-19, was reported for 70,476 (15.9%) patients with an estimated case-fatality ratio of 37.9%; the overall estimated case-fatality ratio is 24.9% (https://www.medrxiv.org/content/10.1101/2020.07.17.20155218v10.full-text).

While several studies reported symptoms and comorbidities associated with severe COVID-19 complications, tools to stratify the risk of developing complications are still lacking. It has been found that changes in plasma proteins offer prognostic molecular profiles that can also identify the most informative clinical features presented at admission to predict the risk of developing complications.

Accordingly, there is a need for better confirmation prognostic tools to identify and predict the risk of severe COVID-19 disease or associated complications.

Such tools may be useful to determine patient populations in need of therapeutic treatment as provided herein.

### SUMMARY

The present disclosure, in part, relates to novel, prognostic tools for severe COVID-19 disease. Furthermore, the present disclosure describes identification of a set of clinical parameters that can be used to generate a clinical risk score of COVID-19 complications.

The present disclosure further relates to proteomic panel-profiling of plasma from patients with severe COVID-19 complications versus mild-moderate symptoms to characterize biological processes and pathways associated with disease severity.

The present disclosure also relates to molecular changes associated with the clinical findings. Furthermore, the present disclosure identifies molecular changes or indicators that can be used to generate a molecular severity score.

Generally, the methods disclosed herein relate to identifying, generating, and using the molecular severity score and/or clinical risk score as prognostic tools for severe COVID-19 disease. In addition, the methods disclosed here relate to identifying effective methods of treatments based on the patient analysis (molecular and clinical identifiers/indicators).

Also provided herein are methods of treating a SARS-CoV-2 infection in a subject in need thereof, comprising administering to the subject a therapeutically effective amount of a drug described herein.

Also provided herein are methods of treating COVID-19 in a subject in need thereof, comprising administering to the subject a therapeutically effective amount of a drug described herein.

Also provided herein are methods of modulating a protein expression profile in a subject in need thereof, comprising administering to the subject an effective amount of a drug described herein, wherein the protein expression profile comprises expression of one or more proteins that are differentially expressed when either 1) compared to a subject currently infected with SARS-CoV-2 as compared to a subject not currently infected with SARS-CoV-2, or 2) compared to a subject currently having COVID-19 as compared to a subject not currently having COVID-19.

In some embodiments, the drug comprises acetylcysteine, adalimumab, alirocumab, alteplase, amiodarone, atenolol, atezolizumab, atorvastatin, bevacizumab, bortezomib, capecitabine, carboplatin, cisplatin, cyclophosphamide, cyclosporine, dexamethasone, diclofenac, didanosine, doxycycline, etanercept, ethoxzolamide, evolocumab, fenofibrate, fentanyl, filgrastim, fluorouracil, flutamide, gemfibrozil, imatinib, indomethacin, infliximab, insulin, irinotecan, lisinopril, lomitapide, lovastatin, mercaptopurine, methotrexate, methylprednisolone, octreotide, oxaliplatin, paclitaxel, paraceramol, paroxetine, pravastatin, prednisone, progesterone, propylthiouracil, raloxifene, ribavirin, rituximab, simvastatin, sirolimus, sorafenib, stavudine, streptozocin, sunitinib, tacrolimus, testosterone, thalidomide, theophylline, vandetanib, verapamil, warfarin, or zidovudine, or combinations thereof.

In some embodiments, the drug comprises anakinra, arsenic trioxide, aspirin, atorvastatin, atropine, bevacizumab, chorionic gonadotropin, citric acid, corticotropin, crizotinib, cyclophosphamide, cyclosporine, dexamethasone, dextrose, doxycycline, eopoetin alfa, epoetin alfa, ethoxzolamide, fluorouracil, flutamide, indomethacin, lopinavir, megestrol acetate, mesalamine, methotrexate, methylene blue, methylprednisolone, octreotide, oxandrolone, paclitaxel, ribavirin, ritonavir, sirolimus, sorafenib, stabudine, stavudine, streptozotocin, sunitinib, tenecteplase, testosterone, thalidomide, theophylline, tretinoin, urokinase, verapamil, vitamin K, zoledronic acid, or zonisamide, or a combination thereof. In some embodiments, the drug is flutamide, lopinavir, mesalamine, methotrexate, methylene blue, ribavirin, ritonavir, or thalidomide, or a combination thereof.

In some embodiments of the methods provided herein, the methods comprise administering to the subject a combination of effective amounts of specific drugs including, but not limited to, ribavirin with infliximab or etanercept and with or without methylprednisolone, ribavirin with methylprednisolone and with or without cyclosporine, or ribavirin with sirolimus and with or without methylprednisolone.

In some embodiments, the drug is anakinra, arsenic trioxide, aspirin, atorvastatin, atropine, bevacizumab, chorionic gonadotropin, citric acid, corticotropin, crizotinib, cyclophosphamide, cyclosporine, dexamethasone, dextrose, doxycycline, eopoetin alfa, ethoxzolamide, fluorouracil, flutamide, indomethacin, megestrol acetate, methylprednisolone, octreotide, oxandrolone, paclitaxel, sirolimus, sorafenib, stabudine, streptozotocin, sunitinib, tenecteplase, testosterone, thalidomide, theophylline, tretinoin, urokinase, verapamil, vitamin K, zoledronic acid, or zonisamide, or a combination thereof. In some embodiments, the drug is anakinra, bevacizumab, citric acid, crizotinib, cyclophosphamide, dexamethasone, dextrose, ethoxzolamide, indomethacin, paclitaxel, sorafenib, sunitinib, or zonisamide, or a combination thereof. In some embodiments, the drug is cyclosporine, fluorouracil, sirolimus, streptozotocin, tenecteplase, or tretinoin, or a combination thereof. In some embodiments, the drug is arsenic trioxide, aspirin, atorvastatin, atropine, chorionic gonadotropin, cyclosporine, doxycycline, epoetin alfa, fluorouracil, flutamide, megestrol acetate, methylprednisolone, octreotide, oxandrolone, sirolimus, stavudine, streptozotocin, tenecteplase, testosterone, thalidomide, theophylline, tretinoin, urokinase, verapamil, vitamin K, or zoledronic acid, or a combination thereof.

### BRIEF DESCRIPTION OF THE DRAWINGS

FIG. 1: Differential protein expression in plasma from patients with active SARS-CoV-2 infection. The limma package was used to identify differentially expressed proteins (DEPs) from the single Olink panels and the combined dataset (893 unique proteins) which was defined as protein with more than 1.25-fold change with a P-value<0.05 and FDR<0.1. (a) Summary of the number of DEPs in each of the ten Olink panels used in the study. DEPs were used to calculate a score for each panel (refer to Methods), which was used for ROC curve analysis and the AUC under the ROC curves is stated for each panel. All ROC curves AUC had a P-value<0.01. DeLong et al. method. (b) Unsupervised hierarchical clustering based on all proteins (a total of 893 unique proteins) assayed using the ten Olink panels showed a separation between patients with severe complications compared to mild cases and controls. The heatmap shows z-scores and clustering was done using correlation and average linkage. Principal component analysis (PCA) confirmed the separation of the severe cases based on the expression profiles of all proteins. (c) Volcano plots summarizing the DEPs across the patient groups. Differential expression analysis addressed severity as the main effect and included all factors, from obesity to SpO2 (except for disease grading), to correct for the interaction of these factors with severity. The time between admission to blood collection was also considered for interaction with disease severity in the comparison between severe and mild cases (right volcano plot in panel c). The number and percentage of the DEPs relevant to all proteins assayed are stated in each panel. Similar analyses were carried out for each panel and shown in FIG. 2.

FIG. 2: Differential protein expression in plasma from patients with active SARS-CoV-2 infection (per panel data). Unsupervised hierarchical clustering based of all 92 proteins in each of the named Olink panel showed a separation between patients with severe complications compared to mild cases and controls. The heatmap shows z-scores and clustering was done using correlation and average linkage. Principal component analysis (PCA) tested the separation of the severe cases based on the expression profiles of all proteins. The limma package was used to identify differentially expressed proteins (DEPs) from the single Olink panels defined as protein with more than 1.25-fold change with a p-value of <0.05 and FDR<0.1. Differential expression analysis addressed severity as the main effect and included obesity, age, sex, ethnicity, heart rate and SpO2 to correct for the interaction of these factors with disease severity. A summary of the number of DEPs for each panel is shown in FIG. 1, panel (a).

FIG. 3: Functional analysis of deregulated plasma proteins in severe versus mild COVID-19 disease. Differentially expressed proteins (DEPs) in patients with severe complications compared to mild-moderate disease were subjected to network analysis using the STRING-db (FIG. 4) and annotation for their function as circulating proteins. Of the 375 DEPs (1.25-fold change in severe vs. mild cases), 288 (77%) DEPs shown in the FIGURE could be allocated to 11 functional groups considering their potential function as circulating proteins; chemotaxis, coagulopathy/fibrinolysis, immune evasion, innate immunity, T- or NK-cell immunity, T-/Th-cells dysfunction, inflammation, neutrophils/neutrophil extracellular traps (NETosis), and organ damage (lung, cardiovascular or other and multiple organs). The remaining 87 DEPs were either known to exist in circulation with unclear function or with known function but with no literature supporting their secretion or release into the blood. The color intensities (red: upregulated, blue: downregulated; legend) depict the log 2 fold-change between severe and mild-moderate cases. DEPs are classified as agonists (pos.) or antagonist (neg.) for the Th1/Th17 and Th2 immune responses. Network interactions between the 278 DEPs and their correlation with clinical blood test are shown in FIG. 4.

FIG. 4: Functional networks of deregulated plasma proteins in severe versus mild COVID-19 disease (network structure). Differentially expressed proteins (DEPs) in patients with severe complications compared to mild-moderate disease were subjected to network analysis using the STRING database and annotation for their function as circulating proteins. Of the 375 DEPs (1.25-fold change in severe vs. mild cases), 288 (77%) DEPs could be allocated to 11 functional groups considering their potential function as circulating proteins; chemotaxis, coagulopathy/fibrinolysis, immune evasion, innate immunity, T- or NK-cell immunity, T-/Th-cells dysfunction, inflammation, neutrophils/neutrophil extracellular traps (NETosis), and organ damage (lung, cardiovascular or other and multiple organs). DEPs are classified as agonists (pos.) or antagonist (neg.) for the Th1/Th17 and Th2 immune responses. The color intensities (red: upregulated, blue: downregulated; legend) depict the log 2 fold-change between severe and mild-moderate cases. Interactions between the 288 DEPs are shown only for those with STRING-db confidence score≥0.7 are shown (587 high-confidence interactions). Inserted table in the FIGURE summarizes the number of interactions across the different STRING-db confidence scores (0.4 to 0.99). The heatmaps summarize the Pearson's correlation coefficient (r) for significant correlations (p<0.05, GraphPad Prism) between each protein in the functional networks and the clinical blood biochemical markers and blood cell counts available in our cohort.

FIG. 5: Correlation between the clinical blood markers and the differentially expressed plasma proteins. The correlation between the expression of the 375 differentially expressed plasma proteins and the available blood markers and blood cell counts in our cohort was evaluated. The overall number (and the percentage) of the DEPs which correlated (significance of p<0.05 using Pearson's correlation, GraphPad Prism) with each clinical measurement are shown in the top row. The heatmap shows the number of DEPs (and the percentage depicted by the heatmap colors) which correlated with each clinical parameter stratified by the functional annotations shown in FIG. 2. CRP showed the highest number of overall and function-specific correlations with the DEPs.

FIG. 6: Drug-protein interactions of upregulated plasma proteins in severe COVID-19 patients. Proteins with more than 2-fold upregulation in severe versus mild-moderate cases were subjected to protein-drug interaction analysis (PDI, using Drug-Gene Interaction database DGIdb, v4.2.0). Target proteins are colored red according to the fold change of expression in severe versus mild cases, whereas drugs are shown in grey boxes or nodes. Drugs that target 1.5- to 2-fold upregulated proteins in severe versus mild cases are shown in FIG. 7. Interactions between proteins are depicted by red or blue lines for STRING-db confidence score of 0.7 to 1.0 or 0.5 to 0.7, respectively. (a) Drugs which target single proteins with 2-fold or more upregulation in severe COVID-19 patients versus mild-moderate cases. (b) Drugs which target with two or more upregulated proteins in severe COVID-19 patients. Those multi-target drugs affect proteins shown in panel b and/or proteins with 1.5- to 2-fold upregulation in severe versus mild cases (FIG. 7).

FIG. 7: Protein-drug interaction network of 1.5- to 2-fold upregulated plasma proteins in severe COVID-19. Proteins with 1.5- to 2-fold upregulation in patients with severe complications versus mild-moderate disease were subjected to protein-drug interaction (PDI) using the Drug-Gene Interaction database (DGIdb, v4.2.0). Target proteins are colored red, and the intensity depicts the fold-change. Drugs which target single proteins are shown in grey boxes and blue font and those that target multiple proteins (on this FIGURE or in FIG. 6) are depicted in black font in blue nodes. Protein-protein interactions are colored according to the STRING-db confidence scores; red: confidence score≥0.7, blue: confidence score 0.5 and <0.7. Drugs in red bold font are notable examples discussed in the main text.

FIG. 8: A signature of 46 plasma proteins can differentiate COVID-19 cases with severe complications versus mild symptoms. (A) MUVR (multivariate modelling with minimally biased variable selection in R) identified the minimum set of proteins as models for patient classifications. Analysis was carried out using all cases, severe and mild cases, mild cases and controls, and severe cases and controls. The number and commonality of the proteins identified from each MUVR analysis is summarized in the Venn diagram. (B) The four modules identified by MUVR and overlap analysis in A were evaluated using receiver-operator characteristic (ROC) analysis. All ROC curves were significantly predictive (p<0.0001). The area under the ROC curves (AUC) is summarized in the panel and the highest AUCs for each comparison (columns) are marked in red font. (C-D) ROC curves based on the 46 DEPs in modules i and ii which were used to calculate the “COVID-19 molecular severity score.” The 46 DEPs were analyzed for PPI and a single, highly interactive network was identified. The enrichment tree summarizes the enriched Gene ontology biological processes (GO-BP) in the molecular severity score. The stated FDR p-values for enrichment are depicted by the size of the red (upregulated) and blue (downregulated) circles.

FIG. 9: A signature of 12 plasma proteins can differentiate COVID-19 cases with severe complications versus mild symptoms. (a) Two variable (feature) selection algorithms were used to select the most robust proteins to differentiate severe cases from mild cases and controls; MUVR (multivariate modelling with minimally biased variable selection in R) and Boruta (a wrapper algorithm for all relevant feature selection and feature importance with random selection runs). Proteins which were shared in the differentiation between patients with severe COVID-19 from the rest of the cohort, and specifically from mild-moderate cases, using MUVR and Boruta were overlapped to select 35 proteins, of which 12 proteins were selected at 100% from 500 random forest runs (Boruta ‘Norm Hits’). (b) Network analysis for the 12 selected proteins showing the STRING-db confidence score. The heatmap summarizes the significant Pearson's correlation coefficients between the 12 selected proteins and clinical blood markers and blood cell counts. (c) ROC curves based on the 12 DEPs which were used to calculate the “COVID-19 molecular severity score” to evaluate the sensitivity, specificity, and the area under the ROC curves (AUC) for differentiating severe COVID-19 cases from mild cases, controls, or both. All ROC curve analyses were significant (p<0.0001 from AUC of 0.5, DeLong et al. method).

FIG. 10: Validation of the 46-protein COVID-19 molecular severity score in the Massachusetts General Hospital (MGH) cohort. (A) The molecular severity scores were calculated based on the expression of the 46 proteins measured using the Olink platform in the MGH cohort. The calculated scores (f SEM) are shown over time for different groups of patients who were followed up for different lengths of time. The percentage change in the scores compared to day 0 was calculated for each patient and the insets summarize the average change (f SEM) over time. (B) Outcomes in the patient groups according to the molecular severity score. All groups were significantly different from each other (Chi-square test p-value was <0.0001 for all comparisons). (C) ROC curves to evaluate the performance of the molecular severity scores were calculated based on data from plasma collected on day 0, day 3 and day 7 (rows). Analysis was carried out for outcomes, i.e. severity. Patients were classified with severe complications if they died or required intubation and ventilation. Mild-moderate cases included hospitalized patients with or without supplementary 02 or who were not hospitalized (discharged). ROC curves for outcomes from day 3 to 28, and the maximum (worst) outcome throughout the study (‘all outcomes’) are shown in the first four columns. ROC curves for death after COVID-19 infection according to the molecular severity scores on different days are shown in the last columns. The sensitivity (sens. %), specificity (spec. %) and the area under the ROC curve (AUC) are shown in each panel. All ROC curves were statistically significant (p<0.0001 from AUC of 0.5).

FIG. 11: Validation of the 12-protein COVID-19 molecular severity score in the Massachusetts General Hospital (MGH) cohort. (a) The molecular severity scores were calculated based on the expression of the 12 proteins measured using the Olink platform in the MGH cohort. The calculated scores (f SEM) are shown over time according to the WHO ordinal scale for COVID-19 severity, acuity groups 1-53, on day 28 after recruitment (left panel) or the maximum acuity over the 28-day study period (right panel). The number of patients in each group is shown in the bar graph. (b) Time curve of the molecular severity scores for the severe COVID-19 groups, A1 group (death) and A2 group (intubated, ventilated but survived 28 days), compared to the remaining groups (A3-A5). * and # in panels a and b denote statistical differences (p<0.01) between the A1 group to the other and the A2 group to the other groups, respectively (two-way ANOVA with Tukey's multiple testing correction). (c) Summary of ROC curve analyses to evaluate the performance of the molecular severity scores on days 0, 3 and 7 in the MGH cohort to predict the maximum COVID-19 severity throughout the 28 day-study. (d-e) Summary of ROC curve analyses to evaluate the performance of the molecular severity scores on day 0 or day 3 to predict COVID-19 severity or death between days 3 to 28 or days 7 to 28, respectively. The AUC, sensitivity (sens.), specificity (spec.), and the number of severe events in each ROC curve are stated. All ROC curves were statistically significant (p<0.0001 from AUC of 0.5, DeLong et al. method).

FIG. 12: A clinical risk score for COVID-19 complications based on the 12-protein molecular severity score. (a) The clinical parameters available in the cohort were evaluated for their association with the 12-protein molecular severity score to identify significantly associated parameters and allow scoring (weighting) for the different groups in each clinical measurement. The box plots show the molecular severity score across the groups in the most associated clinical parameters; the number of patients (total of 100, 50 severe and 50 non-severe) with data available for each parameter is stated in each panel. One-way ANOVA with Dunnett's multiple testing correction was used for clinical parameters with more than two groups, and unpaired two-tailed t-test was used for parameters with two groups. The groups in each of the seven selected clinical parameter (markers) were given a numeric, integer value from 0 to 3 (shown in red bold font) according to the 12-protein severity score. These values were then used to calculate the “Clinical Risk Score” by adding the values across the 7 markers for each patient. Refer to FIG. 13 for details of variable selection. (b) ROC curve analysis confirmed the significant predictive value of the Clinical Risk Score which combined the 7 clinical markers. (c-d) Of the 7 markers in panel a, 4 (CRP and creatinine levels and lymphocyte and neutrophil cell counts) were available in the MGH cohort, thus, were used for independent validation. Panels c and d show the ROC curve of the Clinical Risk Score based on the 4 markers in our cohort from Qatar and the MGH cohort (from day 0 and day 3 data) from the US, respectively. Panel d also shows the risk of COVID-19 severity (% risk with 95% confidence interval) in the MGH cohort according to the 4-marker Clinical Risk Score. For panels b, c and d, the Clinical Risk Scores outperformed each of the single clinical parameters in pairwise comparisons (p<0.0001, DeLong et al. method).

FIG. 13: Comparison of the 12-protein COVID-19 molecular severity score across the groups within the clinical parameters included in the study cohort. (a) Boxplots for the score from the 12-protein signature across the stated groups in each of the clinical annotations in infected patients (n=100). One-way ANOVA with Dunnett's multiple testing correction was used for clinical parameters with more than two groups, and unpaired two-tailed t-test was used for parameters with two groups. Significant differences are depicted as *p<0.05, **p<0.01, ***p<0.001 and ****p<0.0001. (b) ROC curve analysis of the parameters which showed significant association with the 12-protein molecular severity score. The DeLong et al. method was used for statistical analysis. (c) MUVR was used for variable selection using the same parameters in panel b. Seven parameters (markers) were selected by MUVR and the boxplot summarizes the median ranking (with minimum and maximum) from 500 MUVR runs. (d) The model of 7 markers from MUVR was further confirmed for performance using ROC curve analysis in comparison to models which included the remaining clinical markers. There was no additional benefit from addition diabetes, SpO2 and/or eosinophil counts as judged by pairwise comparisons (DeLong et al. method) against the model of the 7 markers alone. Abbreviations; Resp. Rate: Respiratory rate, WBC: white blood cells, CRP: C-reactive protein.

FIG. 14: Functional analysis of differentially expressed proteins in plasma of patients with active SARS-CoV-2 infection. (a-c) Heatmaps of the expression differentially expressed proteins (DEPs) in severe (S) and mild (M) cases and control (C) are shown to the side of enrichment trees of enriched KEGG pathways. Upregulated and downregulated proteins and pathways are shown in red and blue respectively. The p-value for enrichment is depicted by the size of the circles in the enrichment trees. (d) Venn diagrams summarizing the shared and unique upregulated (top) and downregulated (bottom) DEPs. The identities of the proteins in each Venn diagram are shown in Table 5.

FIG. 15: Scoring namogram for the 7-marker risk predictor from clinical parameters. Each patient receives a score for each of the named clinical tests where the score of 0, 1, 2 or 3 is based on the clinical measurement. The final score is calculated as the sum of the scores for each of the 7-markers. A final 7-marker score 4 or less, score between 4 and 7, or a score higher than 7 define low, intermediate or high risk, respectively, to develop severe COVID-19 (require intubation or death).

FIG. 16: Scoring namogram for the 4-marker risk predictor from clinical parameters. Each patient receives a score for each of the named clinical tests where the score of 0, 1, 2 or 3 is based on the clinical measurement. The final score is calculated as the sum of the scores for each of the 4-markers. A final 4-marker score 3 or less, score between 3 and 6, or a score higher than 6 define low, intermediate or high risk, respectively, to develop severe COVID-19 (require intubation or death).

FIG. 17: The COVID-19 molecular severity score on day 0 in the SARS-CoV-2 positive and negative patients in Massachusetts General Hospital (MGH) cohort. The MGH cohort collected plasma samples on day 0 (within 24 hours of admission to the emergency department) from symptomatic patients, of whom 78 patients were found to be negative for SARS-CoV-2. The molecular severity score on day 0 was compared across the different severity levels (acuity max over 28-day period) and between SARS-CoV-2pos and SARS-CoV-2neg patients. The number of patients in each group is shown. Only significant differences are depicted (two-way ANOVA with Tukey's multiple testing correction, GraphPad Prism); **p<0.01, ****p<0.0001.

### DETAILED DESCRIPTION

Some definitions are provided hereafter. Nevertheless, definitions may be located in other sections, and the above header “Definitions” does not mean that such disclosures in other sections are not definitions.

As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including,” “containing” and “having” should all be construed to be inclusive, unless such a construction is clearly prohibited from the context. Further in this regard, these terms specify the presence of the stated features but do not preclude the presence of additional or further features.

Nevertheless, the compositions and methods disclosed herein may lack any element that is not specifically disclosed herein. Thus, a disclosure of an embodiment using the term “comprising” is (i) a disclosure of embodiments having the identified components or steps and also additional components or steps, (ii) a disclosure of embodiments “consisting essentially of” the identified components or steps, and (iii) a disclosure of embodiments “consisting of” the identified components or steps. Any embodiment disclosed herein can be combined with any other embodiment disclosed herein.

The term “and/or” used in the context of “X and/or Y” should be interpreted as “X,” or “Y,” or “X and Y.” Similarly, “at least one of X or Y” should be interpreted as “X,” or “Y,” or “X and Y.”

Where used herein, the terms “example” and “such as,” particularly when followed by a listing of terms, are merely exemplary and illustrative and should not be deemed to be exclusive or comprehensive.

A “subject” or “individual” is a mammal, preferably a human.

All percentages expressed herein are by weight of the total weight of the composition unless expressed otherwise. When reference herein is made to the pH, values correspond to pH measured at about 25° C. with standard equipment, unless expressed otherwise. “Ambient temperature” or “room temperature” is between about 15° C. and about 25° C., and ambient pressure is about 100 kPa.

The terms “COVID-19” and “SARS-CoV-2” may be used interchangeably herein. In some embodiments, “COVID-19” refers to the respiratory disease resulting from infection by the “SARS-CoV-2” virus.

The term “treatment” refers to the application of one or more specific procedures used for the amelioration of a disease. In certain embodiments, the specific procedure is the administration of one or more pharmaceutical agents. “Treatment” of an individual (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset.

An “effective amount” or “therapeutically effective amount” refers to an amount of therapeutic compound, such as a drug described herein, administered to a mammalian subject, either as a single dose or as part of a series of doses, which is effective to produce a desired therapeutic effect. In general, the therapeutically effective amount can be estimated initially either in cell culture assays or in animal models, for example, in non-human primates, mice, rabbits, dogs, or pigs. The animal model may also be used to determine the appropriate concentration range and route of administration. Such information can then be used to determine useful doses and routes for administration in humans.

The term “amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

Various non-exhaustive, non-limiting aspects of compositions according to the present disclosure may be useful alone or in combination with one or more other aspects or embodiments described herein. Without limiting the foregoing description, in one aspect, provided herein are methods of identifying, generating, and using the molecular severity score and/or clinical risk score as prognostic tools for severe COVID-19 disease.

In accordance with a second non-limiting aspect of the present disclosure, which may be used in combination with the first aspect, provided herein are methods of analyzing a biological sample from a patient infected with and/or exposed to COVID-19 and identifying a protein signature that can predict COVID-19 infected patients at higher risk of developing severe complications. In some embodiments, the protein signature is a blood-based protein signature.

In accordance with a third non-limiting aspect of the present disclosure, which may be used in combination with each or any of the above-mentioned aspects, provided herein are methods of generating a clinical risk score that can be used alone or in combination with other prognostic indicators or prognostic signatures to predict severe COVID-19 disease in patients infected with and/or exposed to COVID-19.

In accordance with a fourth non-limiting aspect of the present disclosure, which may be used in combination with each or any of the above-mentioned aspects, provided herein are methods of generating a molecular severity score that can be used alone or in combination with other prognostic indicators or prognostic signatures to predict severe COVID-19 disease in patients infected with and/or exposed to COVID-19.

In accordance with a fifth non-limiting aspect of the present disclosure, which may be used in combination with each or any of the above-mentioned aspects, provided herein are methods of repeating the analysis of the patient exposed to and/or infected with COVID-19 and generating new molecular severity score and/or clinical risk score. This method may be repeated once, twice, or more than twice, across time, in a patient exposed to and/or infected with COVID-19.

In accordance with a sixth non-limiting aspect of the present disclosure, which may be used in combination with each or any of the above-mentioned aspects, provided herein are methods of identifying effective methods of treatments based on the patient analysis (molecular and clinical identifiers/indicators) described herein.

Also provided herein are methods of modulating a protein expression profile in a subject in need thereof, comprising administering to the subject an effective amount of a drug described herein, wherein the protein expression profile comprises expression of one or more proteins that are differentially expressed when either 1) compared to a subject currently infected with SARS-CoV-2 as compared to a subject not currently infected with SARS-CoV-2, or 2) compared to a subject currently having COVID-19 as compared to a subject not currently having COVID-19.

Also provided herein are methods of treating a SARS-CoV-2 infection in a subject in need thereof, comprising administering to the subject a therapeutically effective amount of a drug described herein.

In some embodiments of these methods, the drug comprises acetylcysteine, adalimumab, alirocumab, alteplase, amiodarone, atenolol, atezolizumab, atorvastatin, bevacizumab, bortezomib, capecitabine, carboplatin, cisplatin, cyclophosphamide, cyclosporine, dexamethasone, diclofenac, didanosine, doxycycline, etanercept, ethoxzolamide, evolocumab, fenofibrate, fentanyl, filgrastim, fluorouracil, flutamide, gemfibrozil, imatinib, indomethacin, infliximab, insulin, irinotecan, lisinopril, lomitapide, lovastatin, mercaptopurine, methotrexate, methylprednisolone, octreotide, oxaliplatin, paclitaxel, paraceramol, paroxetine, pravastatin, prednisone, progesterone, propylthiouracil, raloxifene, ribavirin, rituximab, simvastatin, sirolimus, sorafenib, stavudine, streptozocin, sunitinib, tacrolimus, testosterone, thalidomide, theophylline, vandetanib, verapamil, warfarin, or zidovudine, or combinations thereof.

In some embodiments of these methods, the drug comprises anakinra, arsenic trioxide, aspirin, atorvastatin, atropine, bevacizumab, chorionic gonadotropin, citric acid, corticotropin, crizotinib, cyclophosphamide, cyclosporine, dexamethasone, dextrose, doxycycline, eopoetin alfa, epoetin alfa, ethoxzolamide, fluorouracil, flutamide, indomethacin, lopinavir, megestrol acetate, mesalamine, methotrexate, methylene blue, methylprednisolone, octreotide, oxandrolone, paclitaxel, ribavirin, ritonavir, sirolimus, sorafenib, stabudine, stavudine, streptozotocin, sunitinib, tenecteplase, testosterone, thalidomide, theophylline, tretinoin, urokinase, verapamil, vitamin K, zoledronic acid, or zonisamide, or a combination thereof.

In some embodiments of the methods provided herein, the subject has been diagnosed with COVID-19.

In some embodiments of the methods provided herein, the methods comprise administering to the subject a combination of effective amounts of specific drugs including, but not limited to ribavirin with infliximab or etanercept and with or without methylprednisolone, ribavirin with methylprednisolone and with or without cyclosporine, or ribavirin with sirolimus and with or without methylprednisolone.

In some embodiments, the drug is flutamide, lopinavir, mesalamine, methotrexate, methylene blue, ribavirin, ritonavir, or thalidomide, or a combination thereof.

In some embodiments, the drug is anakinra, arsenic trioxide, aspirin, atorvastatin, atropine, bevacizumab, chorionic gonadotropin, citric acid, corticotropin, crizotinib, cyclophosphamide, cyclosporine, dexamethasone, dextrose, doxycycline, eopoetin alfa, ethoxzolamide, fluorouracil, flutamide, indomethacin, megestrol acetate, methylprednisolone, octreotide, oxandrolone, paclitaxel, sirolimus, sorafenib, stabudine, streptozotocin, sunitinib, tenecteplase, testosterone, thalidomide, theophylline, tretinoin, urokinase, verapamil, vitamin K, zoledronic acid, or zonisamide, or a combination thereof.

In some embodiments, the drug is anakinra, bevacizumab, citric acid, crizotinib, cyclophosphamide, dexamethasone, dextrose, ethoxzolamide, indomethacin, paclitaxel, sorafenib, sunitinib, or zonisamide, or a combination thereof.

In some embodiments, the drug is cyclosporine, fluorouracil, sirolimus, streptozotocin, tenecteplase, or tretinoin, or a combination thereof.

In some embodiments, the drug is arsenic trioxide, aspirin, atorvastatin, atropine, chorionic gonadotropin, cyclosporine, doxycycline, epoetin alfa, fluorouracil, flutamide, megestrol acetate, methylprednisolone, octreotide, oxandrolone, sirolimus, stavudine, streptozotocin, tenecteplase, testosterone, thalidomide, theophylline, tretinoin, urokinase, verapamil, vitamin K, or zoledronic acid, or a combination thereof.

In some embodiments, the subject comprises an at least 1.5-fold upregulation, independently, of one or more expressed proteins selected from ACE2, ACP5, ADM, AREG, CA3, CA5A, CALCA, CD274, CD38, CD40, CDH2, CES1, CST3, CTSL, CX3CL1, CXCL10, DCN, DKK1, EPHB4, F3, FAS, FKBP4, FKBP5, FST, GALNT2, GDF15, HGF, HMOX1, HSPB1, IGFBP1, IL10, IL15, IL1R2, IL2RA, IL4R, IL5RA, IL6, IL6R, KLRD1, KRT19, LDLR, LEPR, MAD1L1, MERTK, MME, MMP3, MMP7, MMP9, MPO, NUCB2, PCSK9, PDGFRA, PLAT, PLAUR, PRSS2, REG1A, REN, S100A12, SMPD1, SPP1, SULT2A1, TFF3, TFPI, THBD, THBS2, TNFRSF10A, TNFRSF11A, TNFRSF11B, TNFRSF1A, TNFRSF1B, TYMP, VCAM1, VCAN, or VWF as compared to a corresponding average protein expression from a control cohort of subjects not infected with SARS-CoV-2 or as compared to a corresponding average protein expression from a cohort of SARS-CoV-2 infected subjects with no symptoms or mild-moderate symptoms.

In some embodiments, the at least 1.5-fold upregulation is an at least 2-fold upregulation.

In some embodiments, the subject comprises an at least 1.5-fold downregulation, independently, of one or more expressed proteins selected from downregulated proteins identified herein as compared to a corresponding average protein expression from a control cohort of subjects not infected with SARS-CoV-2 or as compared to a corresponding average protein expression from a cohort of SARS-CoV-2 infected subjects with no symptoms or mild-moderate symptoms.

In some embodiments, the at least 1.5-fold downregulation is an at least 2-fold downregulation.

In some embodiments, the upregulated protein is one or more proteins selected from Table 1. In some embodiments, the downregulated protein is one or more proteins selected from Table 1.

TABLE 1

| | | 46-protein | Severe versus | Severe versus |
|---|---|---|---|---|
| | Direction | signature | mild ratio | control ratio |
| 1 | Up | AREG | 5.35 | 8.47 |
| 2 | Up | HGF | 3.71 | 5.67 |
| 3 | Up | IL18R1 | 1.86 | 2.17 |
| 4 | Up | IL1RL1 | 4.31 | 5.33 |
| 5 | Up | IL6 | 0.87 | 0.27 |
| 6 | Up | KRT19 | 0.66 | 0.38 |
| 7 | Up | PTX3 | 5.23 | 8.88 |
| 8 | Up | SMOC1 | 2.57 | 3.09 |
| 9 | Up | TNC | 2.17 | 1.52 |
| 10 | Up | TNFRSF10B | 0.31 | 0.27 |
| 11 | Down | MSTN | 0.64 | 0.40 |
| 12 | Down | CLEC4C | 0.82 | 0.55 |
| 13 | Up | ACE2 | 4.32 | 8.04 |
| 14 | Up | ADM | 2.76 | 3.14 |
| 15 | Up | ANGPTL1 | 4.32 | 6.83 |
| 16 | Up | CCL7 | 4.01 | 5.03 |
| 17 | Up | CDH2 | 1.75 | 2.24 |
| 18 | Up | CEACAM8 | 3.66 | 4.93 |
| 19 | Up | CTSL | 2.20 | 2.70 |
| 20 | Up | GFRA1 | 4.37 | 5.98 |
| 21 | Up | HAVCR1 | 3.08 | 3.65 |
| 22 | Up | HS3ST3B1 | 2.91 | 2.61 |
| 23 | Up | IGFBP2 | 9.64 | 12.04 |
| 24 | Up | IL15 | 18.44 | 30.07 |
| 25 | Up | IL17RB | 3.14 | 3.21 |
| 26 | Up | IL1R2 | 0.82 | 0.61 |
| 27 | Up | IL6R | 0.38 | 0.26 |
| 28 | Up | LRIG1 | 8.36 | 12.20 |
| 29 | Up | MATN3 | 1.46 | 2.21 |
| 30 | Up | NECTIN2 | 6.64 | 9.02 |
| 31 | Up | PDGFRA | 0.40 | 0.24 |
| 32 | Up | PRTN3 | 1.93 | 2.47 |
| 33 | Up | PTN | 0.78 | 0.58 |
| 34 | Up | PTS | 2.44 | 2.30 |
| 35 | Up | PVR | 2.89 | 2.88 |
| 36 | Up | SIGLEC10 | 13.23 | 28.50 |
| 37 | Up | TINAGL1 | 4.97 | 8.31 |
| 38 | Down | COMP | 4.97 | 7.30 |
| 39 | Down | CRTAC1 | 2.62 | 3.60 |
| 40 | Down | BANK1 | 0.85 | 0.64 |
| 41 | Down | BMP4 | 2.49 | 2.78 |
| 42 | Down | KIT | 0.27 | 0.19 |
| 43 | Down | KITLG | 8.57 | 13.83 |
| 44 | Down | NTRK2 | 2.58 | 3.23 |
| 45 | Down | RGMA | 4.82 | 6.00 |
| 46 | Down | SKAP1 | 3.38 | 4.85 |

In some embodiments, the upregulated protein is one or more proteins selected from Table 2. In some embodiments, the downregulated protein is one or more proteins selected from Table 2.

TABLE 2

| | | 12-protein | Severe versus | Severe versus |
|---|---|---|---|---|
| | Direction | signature | mild ratio | control ratio |
| 1 | Up | AREG | 5.35 | 8.47 |
| 2 | Up | HGF | 3.71 | 5.67 |
| 3 | Up | IL18R1 | 1.86 | 2.17 |
| 4 | Up | IL1RL1 | 4.31 | 5.33 |
| 5 | Up | IL6 | 0.87 | 0.27 |
| 6 | Up | KRT19 | 0.66 | 0.38 |
| 7 | Up | PTX3 | 5.23 | 8.88 |
| 8 | Up | SMOC1 | 2.57 | 3.09 |
| 9 | Up | TNC | 2.17 | 1.52 |
| 10 | Up | TNFRSF10B | 0.31 | 0.27 |
| 11 | Down | MSTN | 0.64 | 0.40 |
| 12 | Down | CLEC4C | 0.82 | 0.55 |

In some embodiments, the subject comprises a molecular severity score of at least 20, wherein the molecular severity score is calculated by the average expression of 10 proteins (IL6, IL1RL1, SMOC1, KRT19, PTX3, TNC, AREG, HGF, TNFRSF10B, IL18R1), divided by the average expression of 2 proteins (MSTN and CLEC4C).

In some embodiments, the subject comprises a molecular severity score of at least 15,
wherein the molecular severity score is by the average expression of 35 proteins (ACE2, ADM, ANGPTL1, AREG, CCL7, CDH2, CEACAM8, CTSL, GFRA1, HAVCR1, HGF, HS3ST3B1, IGFBP2, IL15, IL17RB, IL18R1, IL1R2, IL1RL1, IL6, IL6R, KRT19, LRIG1, MATN3, NECTIN2, PDGFRA, PRTN3, PTN, PTS, PTX3, PVR, SIGLEC10, SMOC1, TINAGL1, TNC, TNFRSF10B), divided by the average expression of 11 proteins (BANK1, BMP4, CLEC4C, COMP, CRTAC1, KIT, KITLG, MSTN, NTRK2, RGMA, SKAP1).

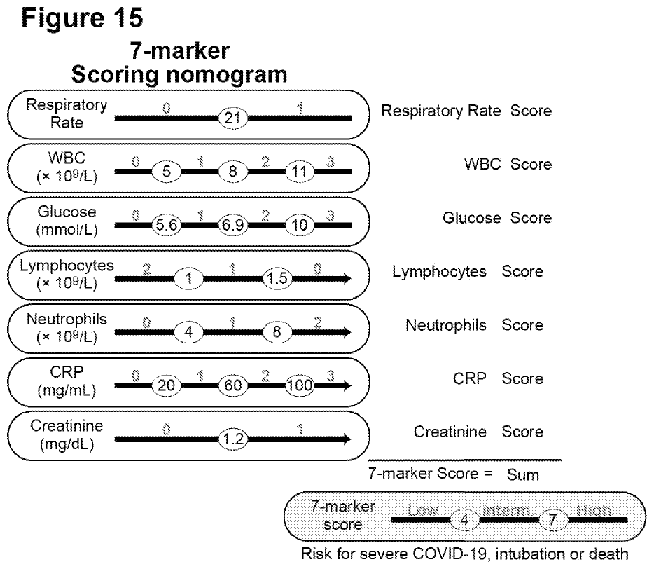

In some embodiments, the subject comprises a clinical score of at least 7, wherein the clinical score is calculated by the sum of scores for respiratory rate, whole blood cells count, glucose concentration, lymphocyte counts, Neutrophil counts, CRP level and Creatinine level as detailed in the 7-marker nomogram (FIG. 15).

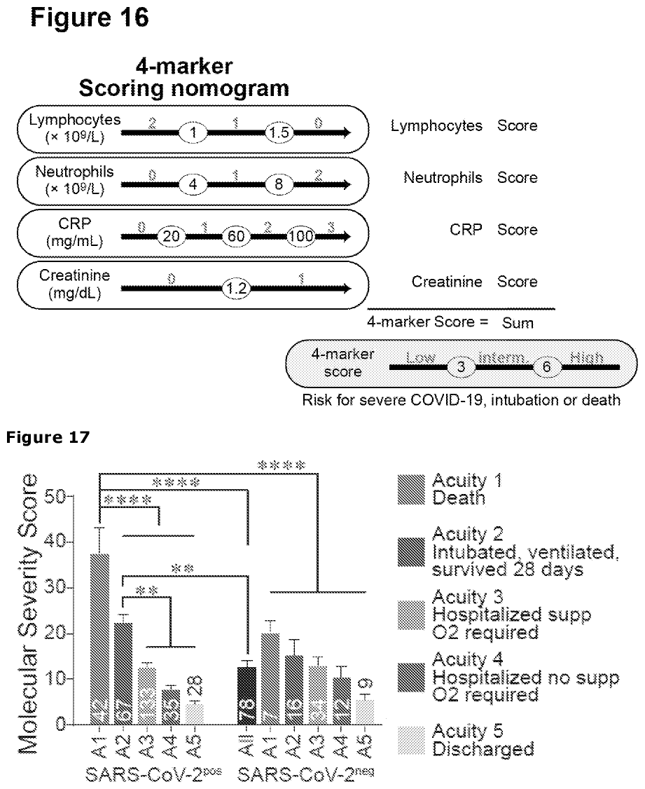

In some embodiments, the subject comprises a clinical score of at least 6, wherein the clinical score is calculated by the sum of scores for Lymphocyte counts, Neutrophil counts, CRP level and Creatinine level as detailed in the 7-marker nomogram (FIG. 16).

In some embodiments, the subject has been admitted to a hospital not more than about three days prior to administering the drug.

In some embodiments, the subject comprises one or more of the comorbidities listed in Table 4.

### Examples

COVID-19 complications still present a huge burden on healthcare systems and warrant predictive risk models for disease severity to enable triaging of patients and early intervention. We profiled 893 plasma proteins from COVID-19 patients (severe complication n=50, and mild-moderate symptoms n=50) and a healthy controls group (n=50). The patterns of dysregulation of 375 plasma proteins in severe patients were deconvoluted based on functions, particularly in circulation, to gain biological insight into the pathogenesis of severe COVID-19. Additionally, we proposed candidate FDA-approved drugs that target multiple upregulated plasma proteins to treat severe complications. We also developed a robust 12-plasma protein signature and a model that combines seven routine clinical tests available at admission, which were validated in an independent cohort as early risk predictors of severity and outcomes. The risk predictors and candidate drugs described in our study can be used and developed for personalized management of SARS-CoV-2 infected patients.

The rapid and widespread dissemination of the severe acute respiratory syndrome coronavirus 2 (SARS-CoV-2) has pressured healthcare systems globally. To date, there have been over 60 million individuals infected worldwide, leading to over 1.5 million deaths due to severe complications from the Coronavirus disease 2019 (COVID-19). The International Severe Acute Respiratory and Emerging Infections Consortium (ISARIC) released its latest comprehensive report on 14 Jul. 2021 including data from 30 Jan. 2020 to 25 May 2021 for 442,643 individuals with laboratory-confirmed SARS-CoV-2 infections from more than 1,600 sites across 61 countries. Patients were split equally between males (221,591) and females (220,390), with a median age of 60 years. The most common comorbidities at admission were hypertension (41%), smoking (35%), diabetes mellitus (28%), cardiovascular disease (17%), and obesity (12%)1. The five most common symptoms at admission were shortness of breath, cough, history of fever, fatigue, and altered consciousness or confusion. Oxygen saturation (SpO2%) less than 94% was present in 34.8% and 25.3% of the patients who were and were not on oxygen therapy at admission, respectively. Admission to intensive care or high dependency units (ICU/HDU) at some point of illness, which could be defined as severe COVID-19, was reported for 70,476 (15.9%) patients with an estimated case-fatality ratio of 37.9%; the overall estimated case-fatality ratio is 24.9%1.

Several studies reported symptoms and comorbidities associated with severe COVID-19 complications; however, early prognostic tools to stratify the risk of developing complications are imperative. In this study, we hypothesized that changes in plasma proteins offer prognostic molecular profiles and can help identify the most informative clinical features presented at admission, which can predict the risk of developing complications. To address this, we used proteomic panel-profiling of plasma from patients with severe complications versus mild-moderate symptoms and control subjects to characterize biological processes and pathways associated with disease pathogenesis and severity. Then we evaluated the plasma proteins and associated routine clinical tests in an independent cohort and examined candidate FDA-approved drugs targeting multiple upregulated proteins and based on biological pathways specific for patients with severe complications.

Results

Study Cohort Characteristics

Characteristics of the study groups, patients (severe and mild-moderate) and healthy controls, are summarized in Table 3. Most infected patients were males (n=91, 91%). The median age [interquartile range (IQR)] of patients with severe COVID-19 disease defined by admission to ICU (47[35-55] years), but not mild-moderate patients, was higher than the control groups (vs. 38[33-42] years, p<0.001). The ethnicity distribution in the severe and mild-moderate groups was not significantly different; however, the control group had a higher percentage of the Indian subcontinent ethnicity (p=0.04). Patients with severe disease had a significantly higher BMI and were either overweight (n=25, 50%) or obese (n=18, 36%) (p<0.001), and had a significantly higher heart rate and lower SpO2 (p<0.001 for both). Moreover, diabetes and hypertension were significantly associated with severe complications in the lungs and kidneys, compared to mild-moderate disease (Table 4).

TABLE 3
Characteristics of patients with COVID-19 and controls.

| | | | | | |
|---|---|---|---|---|---|
| | | Mild- | | | |
| | Controls | Moderate | Severe | Total | p- |
| Variables | (N = 50) | (N = 50) | (N = 50) | (N = 150) | value |

Age (years)

| | | | | | |
|---|---|---|---|---|---|
| Mean + SD | 37.4 ± 7.7 | 40.0 ± 11.9 | 45.9 ± 11.2 | 41.1 ± 0.9 | <0.001 |
| Median [IQR] | 38[33- | 40[32- | 47[35- | 40[34- | |
| | 42] | 51] | 55] * | 49] | |

Sex n (%)

| | | | | | |
|---|---|---|---|---|---|
| F | 2(4.0) | 8(16.0) | 1(2.0) | 11(7.3) | 0.015 |
| M | 48(96.0) | 42(84.0) | 49(98.0) | 139(92.7) | |

Ethnicity n (%)

| | | | | | |
|---|---|---|---|---|---|
| Indian | 43(86.0) | 30(60.0) | 33(66.0) | 106(70.7) | 0.035 |
| subcontinent | | | | | |
| Middle East | 5(10.0) | 15(30.0) | 10(20.0) | 30(20.0) | |
| North Africa | | | | | |
| Others | 2(4.0) | 5(10.0) | 7(14.0) | 14(9.3) | |

BMI (kg/m²)

| | | | | | |
|---|---|---|---|---|---|
| Mean ± SD | 25.4 ± 4.0 | 26.5 ± 3.9 | 29.7 ± 6.1 | 27.2 ± 0.4 | <0.001 |
| Median [IQR] | 24[23- | 26[23- | 28[26- | 26[24- | |
| | 27] | 28] | 33] *^{‡} | 29] | |

Obesity Level n (%)

| | | | | | |
|---|---|---|---|---|---|
| Normal (≤25) | 28(56.0) | 20(40.0) | 7(14.0) | 55(36.7) | <0.001 |
| Overweight (25-30) | 17(34.0) | 21(42.0) | 25(50.0) | 63(42.0) | |
| Obese (30+) | 5(10.0) | 9(18.0) | 18(36.0) | 32(21.3) | |

Heart Rate (beats per minute)

| | | | | | |
|---|---|---|---|---|---|
| Mean ± SD | 76.5 ± 10.0 | 89.8 ± 16.1 | 102.4 ± 16.3 | 89.3 ± 1.5 | <0.001 |
| Median [IQR] | 78[70- | 86[78- | 100[88- | 86[78- | |
| | 82] | 104 ^{‡} | 117] *^{‡} | 100] | |

SpO₂ (%)

| | | | | | |
|---|---|---|---|---|---|
| Mean ± SD | 98.7 ± 0.8 | 98.2 ± 2.1 | 93.6 ± 6.9 | 79.2 ± 0.8 | <0.001 |
| Median [IQR] | 99[98- | 99[97- | 96[91- | 78[72- | |
| | 99] | 100] ^{‡} | 97] *^{‡} | 86] | |

SBP (mmHg)

| | | | | | |
|---|---|---|---|---|---|
| Mean ± SD | 122.5 ± 10.3 | 131.1 ± 17.7 | 125.5 ± 17.6 | 126.4 ± 1.3 | 0.022 |
| Median [IQR] | 121[116- | 129[119- | 128[109- | 126[116- | |
| | 130] | 139] * | 137] | 135] | |

DBP (mmHg)

| | | | | | |
|---|---|---|---|---|---|
| Mean ± SD | 77.9 ± 7.4 | 80.2 ± 9.1 | 79.5 ± 12.6 | 79.2 ± 0.8 | 0.503 |
| Median [IQR] | 78[73- | 81[74- | 78[71- | 78[72- | |
| | 81] | 88] | 86] | 86] | |

BMI, body mass index; SBP, systolic blood pressure; DBP, diastolic blood pressure
* Significantly different than controls
^{‡} Significantly different than Mild-Moderate

TABLE 4
Data of comorbidity for patients admitted to hospital
with Mild-Moderate vs Severe Covid-19 during the
disease peak period of April 2020-May 2020.

| | | Mild- | | | |
|---|---|---|---|---|---|
| | | Moderate | Severe | Total | |
| | | (N = | (N = | (N = | p- |
| Variables n (%) | | 50) | 50) | 100) | value |
| Diabetes | Yes | 6(18.8) | 26(81.3) | 32 | <0.001 |
| mellitus (DM) | No | 44(64.7) | 24(35.3) | 68 | |
| Hypertension | Yes | 12(33.3) | 24(66.7) | 36 | 0.012 |
| | No | 38(59.4) | 26(40.6) | 64 | |
| Smoker | Ex- | 2(50.0) | 2(50.0) | 4 | 0.88 |
| | smoker | | | | |
| | Current | 6(46.2) | 7(53.8) | 13 | |
| | smoker | | | | |
| | Unknown | 18(46.2) | 21(53.8) | 39 | |
| | Non | 24(54.5) | 20(45.5) | 44 | |
| | smoker | | | | |
| Dyslipidemia | Yes | 4(36.4) | 7(63.6) | 11 | 0.338 |
| | No | 46(51.7) | 43(48.3) | 89 | |
| Metabolic | Yes | 0(0.0) | 3(100.0) | 3 | 0.242 |
| Syndrome | No | 50(51.5) | 47(48.5) | 97 | |
| Allergy | Yes | 3(37.5) | 5(62.5) | 8 | 0.715 |
| | No | 47(51.1) | 45(48.9) | 92 | |
| Endocrine | Yes | 2(66.7) | 1(33.3) | 3 | 0.999 |
| Disorders | No | 48(49.5) | 49(50.5) | 97 | |
| Kidney | Yes | 1(25.0) | 3(75.0) | 4 | 0.617 |
| | No | 49(51.0) | 47(49.0) | 96 | |
| Gastroen- | Yes | 2(66.7) | 1(33.3) | 3 | 0.999 |
| terology | No | 48(49.5) | 49(50.5) | 97 | |
| Neurology | Yes | 2(100.0) | 0(0.0) | 2 | 0.495 |
| | No | 48(49.0) | 50(51.0) | 98 | |
| Coronary artery | Yes | 1(25.0) | 3(75.0) | 4 | 0.617 |
| disease (CAD) | No | 49(51.0) | 47(49.0) | 96 | |
| Malignancy | Yes | 1(100.0) | 0(0.0) | 1 | 0.999 |
| | No | 49(49.5) | 50(50.5) | 99 | |

Affected organs

| | | | | | |
|---|---|---|---|---|---|
| Lung | Yes | 25(33.8) | 49(66.2) | 74 | <0.001 |
| | No | 25(96.2) | 1(3.8) | 26 | |
| Kidney | Yes | 0(0.0) | 11(100.0) | 11 | <0.001 |
| | No | 50(56.2) | 39(43.8) | 89 | |
| Sepsis & Multi | Yes | 0(0.0) | 8(100.0) | 8 | 0.006 |
| Organ Failure | No | 50(54.3) | 42(45.7) | 92 | |
| Liver | Yes | 6(33.3) | 12(66.7) | 18 | 0.118 |
| | No | 44(53.7) | 38(46.3) | 82 | |
| Heart | Yes | 0(0.0) | 2(100.0) | 2 | 0.495 |
| | No | 50(51.0) | 48(49.0) | 98 | |

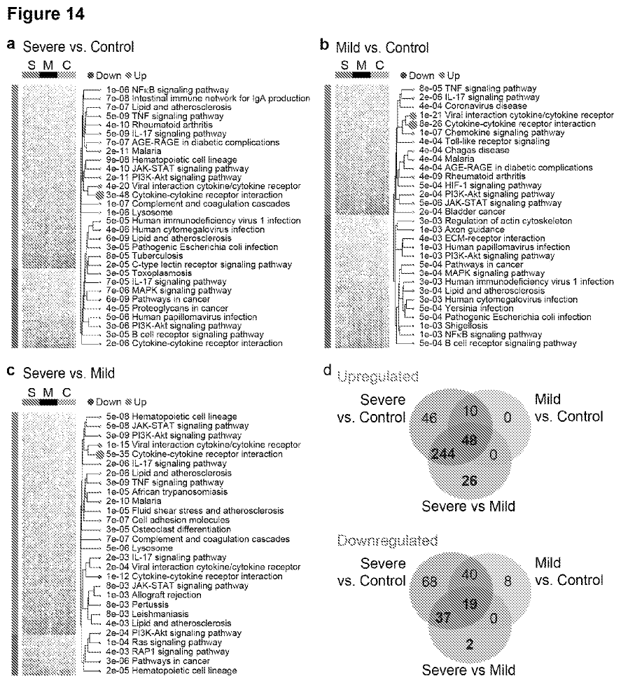

TABLE 5
Differentially expressed proteins from FIG. 14,
panel (d). Unique proteins for each comparison.
Upregulated Proteins

| | |
|---|---|
| Severe vs. | ADGRE2, ALDH3A1, ANGPTL3, BAMBI, CA12, CANT1, CCL14, |
| Control (46 | CCL18, CCL2, CCL27, CCL4, CD164, CD79B, CLEC1B, COL18A1, |
| proteins) | CXCL8, EGFL7, ENTPD5, ENTPD6, FCRL2, GCNT1, GGT5, HNMT, |
| | IL12RB1, IL15RA, IL17C, IL32, ITM2A, LAG3, LEP, LRP11, LTA4H, |
| | LTBP3, MBL2, NPDC1, OSCAR, PDCD1LG2, PDGFC, PIGR, PTK7, |
| | RARRES2, SCGB3A2, SERPINA12, TNFSF13B, TXNDC5, WNT9A |
| Severe vs. Mild | AHCY, ALCAM, AZU1, BAG6, CDH1, CDSN, DFFA, F11R, FCGR2A, |
| (26 proteins) | FCGR3B, HMOX2, IL22RA1, ITGB6, LCN2, LTBP2, PLAU, PLXNB3, |
| | PVALB, RNASE3, SELE, SELP, SLITRK2, CD93, LYVE1, NMNAT1, |
| | NPM1 |
| Mild vs. Control | |
| (0 proteins) | |

Downregulated Proteins

| | |
|---|---|
| Severe vs. | AARSD1, AKT1S1, ANXA4, APBB1IP, APEX1, APOM, ARG1, |
| Control (68 | ATG4A, BACH1, BCAN, BCR, CAPG, CASP3, CASP8, CBL, CBLIF, |
| proteins) | CCL17, CD244, CD2AP, CDH17, CDH5, CETN2, CNTN2, COL1A1, |
| | CRADD, DPEP1, EDAR, EIF4B, ERBIN, FABP2, FABP9, FLI1, |
| | FOXO1, GLO1, GPNMB, GRAP2, HCLS1, HPGDS, IL16, IRAK4, |
| | IRF9, ITGA6, KAZALD1, KIF1BP, MAP2K6, MAX, MDGA1, NFKBIE, |
| | NTRK3, NUB1, PIK3AP1, PRDX5, PRKRA, PRTFDC1, PXN, RGMB, |
| | RPS6KB1, S100P, SERPINA9, SH2D1A, STX8, TAFA5, TBCB, |
| | TDRKH, TGM2, THBS4, TMPRSS5, WWP2 |
| Severe vs. Mild | FETUB, GPC5 |
| (2 proteins) | |
| Mild vs. Control | CD93, CEACAM8, DECR1, LYVE1, NMNAT1, NPM1, PRKCQ, |
| (8 proteins) | SCARF2 |

TABLE 6
Differentially expressed proteins from FIG. 14,
panel (d). Common proteins for each comparison.
Upregulated Proteins

| | |
|---|---|
| Common | ACE2, ACP5, ADM, CCL7, CD274, CFHR5, CSF1, CTSL, CXCL10, |
| proteins for | CXCL11, DKK1, EBI3/IL27, FBP1, GDF15, GRN, HAVCR1, |
| Severe vs. | HS3ST3B1, IFNL1, IGLC2, IL10, IL2RA, KLB, LAMP3, LGALS9, |
| Control, Severe | LILRB4, LRIG1, MATN3, MERTK, NID1, PTN, PTS, PTX3, PVR, |
| vs. Mild and Mild | SIGLEC1, SMOC1, SMPD1, SOST, SPON2, ST3GAL1, ST6GAL1, |
| vs. Control (48 | TCN2, TIMP1, TIMP4, TINAGL1, TNFRSF10A, TNFRSF10B, TYMP, |
| proteins) | VWF |
| Severe vs. Mild | ACP6, ACVRL1, ADGRG1, AGER, AGR2, AGR3, AGRP, ALDH1A1, |
| and Severe vs. | ANGPT2, ANGPTL1, ANPEP, ANXA10, AOC1, AREG, ASGR1, AXL, |
| Control (244 | BLMH, BST2, BTN3A2, C1QTNF1, C2, CA3, CA5A, CALCA, |
| proteins) | CCDC80, CCL15, CCL20, CCL23, CCL3, CD163, CD300C, |
| | CD300LF, CD302, CD38, CD40, CD59, CD63, CDCP1, CDH2, |
| | CES1, CFC1, CHI3L1, CKAP4, CLEC1A, CLEC4D, CLEC5A, |
| | CLEC6A, CLEC7A, CLSTN2, COL4A1, CPA1, CPB1, CSF3, CST3, |
| | CSTB, CTSC, CTSD, CTSH, CTSO, CTSZ, CX3CL1, CXADR, |
| | CXCL16, CXCL9, DCBLD2, DCN, DDAH1, DDC, DEFA1, DKK4, |
| | DPP7, DRAXIN, EDA2R, EFNA4, ENAH, ENPP7, EPHB4, EZR, F11, |
| | F2R, F3, FABP4, FAM3C, FAS, FCAR, FKBP4, FKBP5, FOSB, FRZB, |
| | FST, GALNT10, GALNT2, GAS6, GFRA1, HAO1, HEXIM1, HGF, |
| | HMOX1, HS6ST1, HSD11B1, HSPB1, ICAM1, ICAM2, IDUA, IFI30, |
| | IFNLR1, IGFBP1, IGFBP2, IGFBP7, IGFBPL1, IGSF3, IL15, |
| | IL17RA, IL17RB, IL18BP, IL18R1, IL1R1, IL1R2, IL1RL1, IL1RL2, |
| | IL4R, IL5RA, IL6, IL6R, IL7, KLRD1, KRT19, KYNU, LAYN, LDLR, |
| | LEPR, LGALS3, LGALS4, LGALS8, LIFR, LILRA5, LILRB1, LILRB2, |
| | LRP1, LRPAP1, LTBR, MAD1L1, MB, MCFD2, MET, MILR1, MME, |
| | MMP10, MMP3, MMP7, MMP9, MPO, MSR1, NADK, NECTIN2, |
| | NEFL, NGF, NOMO1, NOS3, NPPB, NRP2, NT-proBNP, NUCB2, |
| | OSM, PAPPA, PCDH17, PCSK9, PDGFRA, PFDN2, PGF, PGLYRP1, |
| | PHOSPHO1, PILRB, PLAT, PLAUR, PLIN1, PLXNB1, PLXNB2, |
| | PODXL2, PON2, PPP3R1, PREB, PRELP, PROC, PROK1, PRSS2, |
| | PRSS8, PRTN3, QDPR, REG1A, REG4, REN, RETN, RSPO1, |
| | S100A12, SCARB2, SEMA4C, SFRP1, SFTPD, SIGLEC10, |
| | SIGLEC9, SIRPA, SLAMF7, SLAMF8, SNCG, SOD2, SORT1, |
| | SPARCL1, SPOCK1, SPON1, SPP1, STC1, SULT2A1, TACSTD2, |
| | TDGF1, TFF2, TFF3, TFPI, TGFA, TGFBI, THBD, THBS2, THOP1, |
| | TIMD4, TNC, TNFRSF11A, TNFRSF11B, TNFRSF12A, TNFRSF13B, |
| | TNFRSF14, TNFRSF1A, TNFRSF1B, TNNI3, TREM1, TRIM21, |
| | ULBP2, VAMP5, VCAM1, VCAN, VSIG2, VSTM1, ZBTB16, ZBTB17, |
| | CEACAM8 |
| Severe vs. | CCL8, EREG, GH1, HBEGF, MMP1, SAA4, SERPINE1, TGFB1, TNF, |
| Control and Mild | VEGFA |
| vs. Control (10 | |
| proteins) | |

Downregulated Proteins

| | |
|---|---|
| Common | BMP4, BOC, CD1C, CLEC4A, CNTN5, COMP, CR2, DNER, DSG3, |
| proteins for | DSG4, GDF2, ITGA11, KITLG, LRRN1, MSTN, NTRK2, PLXDC1, |
| Severe vs. | ROBO2, USP8 |
| Control, Severe | |
| vs. Mild and Mild | |
| vs. Control (19 | |
| proteins) | |
| Severe vs. Mild | ADAM22, ANXA11, BID, CCL11, CD5, CD6, CLEC10A, CLEC4C, |
| and Severe vs. | CRH, CXCL5, DLK1, EGLN1, FGF19, FLT3LG, GFRA3, GZMA, |
| Control (37 | IGFBP3, IL12A/IL12B, KLK10, LTA, MMP12, NCF2, PAG1, PON3, |
| proteins) | PRSS27, RASSF2, SIGLEC6, SIT1, SKAP1, SSC4D, STK4, |
| Severe vs. | SULT1A1, TNFRSF9, TNFSF10, TNFSF11, TNFSF12, TSHB |
| Control and Mild | ADAM23, ARHGEF12, AXIN1, BANK1, CA13, CA14, CAMKK1, |
| vs. Control (40 | CDH6, CNTN1, CRTAC1, DAPP1, DCTN1, FGR, IKBKG, IKZF2, |
| proteins) | INPPL1, ITGB2, ITGB7, KIT, LAT2, LYAR, MAP4K5, NBN, NFATC1, |
| | NINJ1, NPTXR, OLR1, PLXNA4, PMVK, PPP1R9B, RGMA, SH2B3, |
| | SNAP23, SRC, SRPK2, SUMF2, TANK, TRAF2, WASF1, YES1 |

High differential protein expression in plasma from patients with severe complications.

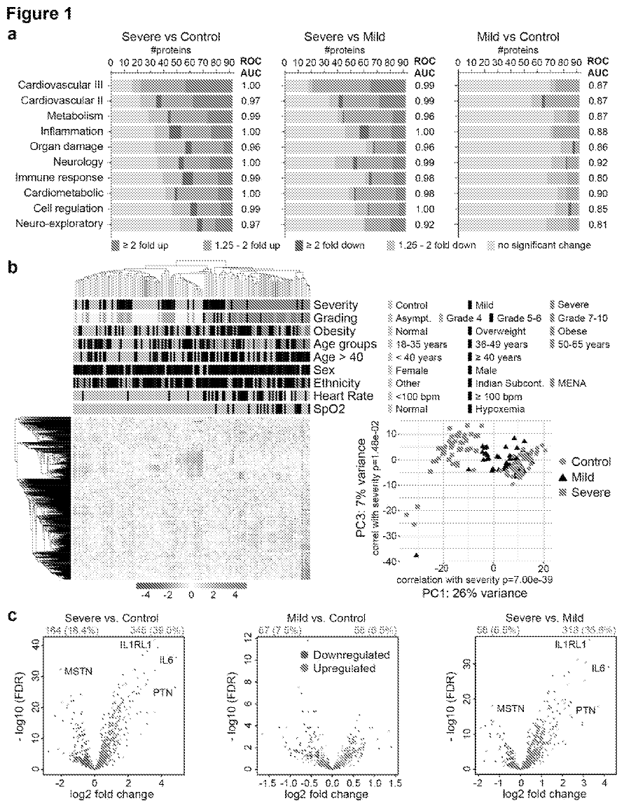

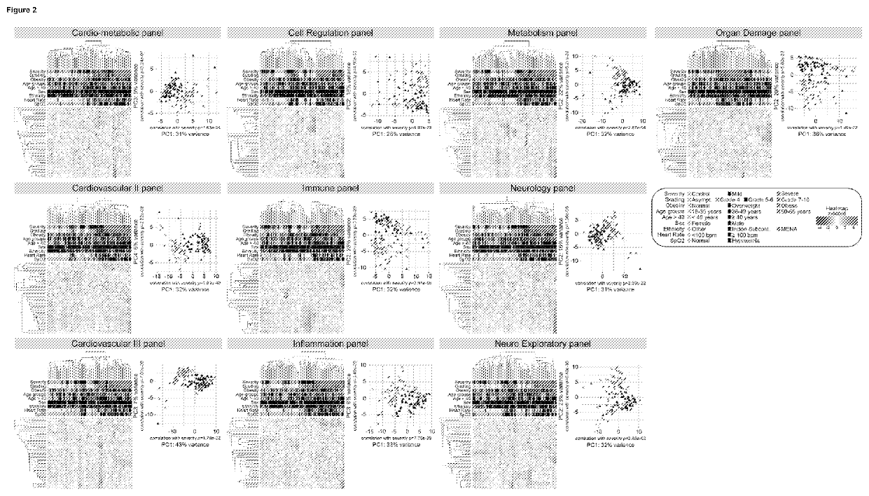

Plasma from 50 severe and 50 with mild-moderate COVID-19 patients and 50 control subjects were analyzed using ten different Olink panels. For one patient, P064, Olink assays failed QC for seven panels; thus, was excluded. The number of differentially expressed proteins (DEPs) from single panels for samples that passed Olink's QC (FIG. 2) is summarized in FIG. 1a. Given the characteristics of our cohort, such as over-representation of males in all groups and younger age of controls, the DEP analyses were corrected for interaction between severity and obesity, sex, age, ethnicity, heart rate, and SpO2. Severe disease versus control identified a large numbers of DEPs; more than 40 out of 92 (>43%) per panel across all panels, whereas the number of DEPs was less in mild-moderate disease versus control. Receiver operating characteristic (ROC) curve analyses using the DEPs in each panel, calculated as a single score, found that all panels significantly classified severe cases versus mild-moderate cases and controls; high area under the curve (AUC, p<0.01) (FIG. 1a).

For a comprehensive molecular view, we carried out the analysis on combined data from the ten Olink panels (893 unique proteins) as a single dataset. Unsupervised hierarchical clustering, before filtering, revealed that the ten panels could differentiate severe from mild-moderate diseases and controls (FIG. 1b). More DEPs were identified when comparing the severe disease to mild-moderate disease or controls than the mild-moderate disease to controls (FIG. 1c). Additionally, the DEPs in severe disease versus mild-moderate disease and controls were mainly upregulated, whereas DEPs in mild-moderate versus controls groups had an equal up-and-down-regulation distribution (FIG. 1c).

Functional analysis of the deregulated proteins in plasma of severe COVID-19 patients.

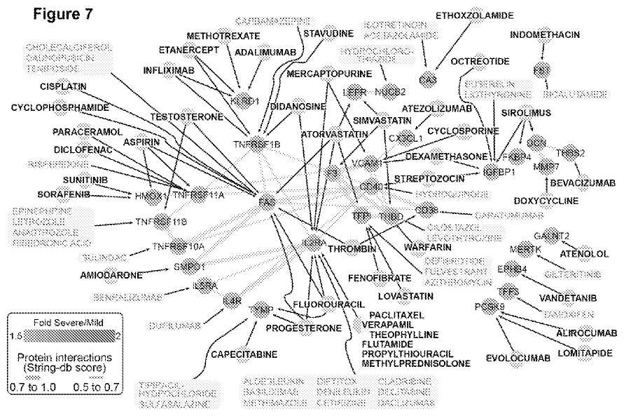

The DEPs in severe disease versus mild disease and controls, and mild versus controls, were subjected to KEGG pathways enrichment analysis. The statistical significance of enriched pathways should be treated cautiously since our proteomic assays were based on enriched panels consisting of 894 unique proteins; however, relative enrichment is warranted. The enrichment of cytokine-cytokine receptor interaction increased gradually from control subjects to patients with mild to severe diseases (FIGS. 7A-C). Such gradual enrichments were observed for several pathways that mainly relate to immune, inflammation and infection-related pathways, and the associated cell signaling pathways. The overlap of the DEPs in the three comparisons found 72 upregulated and 22 downregulated proteins (FIG. 7D). Additionally, 16 and 56 upregulated and downregulated proteins, respectively, were common in severe and mild-moderate disease compared to controls. Protein-protein interaction (PPI) analysis of these common 166 DEPs revealed that they are highly connected with an average of 11 connections per protein (FIG. 7E).

Next, we focused on the differences between patients with severe versus mild-moderate disease. To dissect the differences between these two groups, significantly different DEPs between mild-moderate cases versus controls were excluded. As shown in FIG. 2A, most of the 106 DEPs in severe versus mild disease were also significantly deregulated in severe disease versus controls and were highly connected based on PPI analysis (average 13 connections per protein). The PPI analysis revealed a clear pathway starting from cytokine-cytokine interaction and chemokine signaling, particularly IL-17 signaling, to downstream signaling pathways including TNF, NFKB, Jak-STAT, PI3K, Ras and Rap1 signaling (FIG. 2B). Three additional pathways were also identified in the analysis including complement and coagulation, cell adhesion, and apoptosis and lysosomal degradation.

Potential drugs to target deregulated proteins in COVID-19 patients with severe complication.

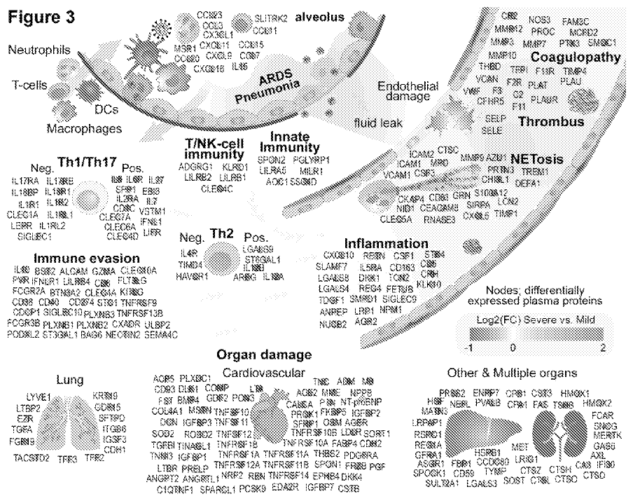

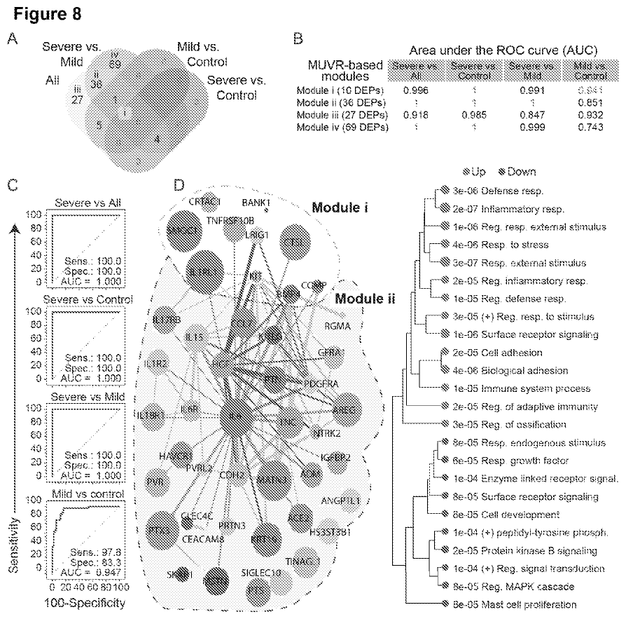

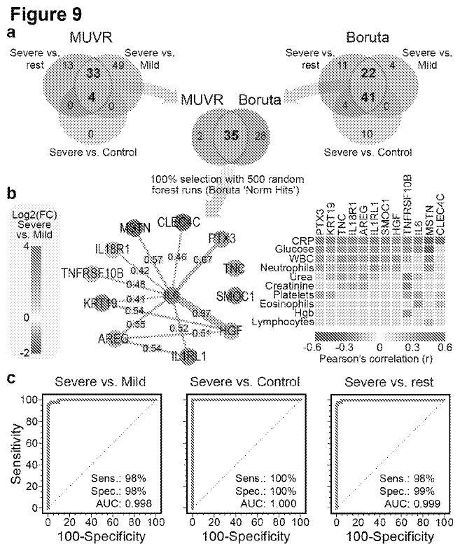

In addition to targeting the enriched pathways (FIG. 2 and FIGS. 7A-E) such as TNFα, coagulation or Jak-STAT, an analysis of protein-drug interaction (PDI) was carried out based on the upregulated proteins in patients with severe versus mild disease. A library of FDA-approved drugs was screened for interactions with 1.5 to 2-fold upregulated proteins (FIG. 8) and >2-fold upregulated proteins (FIG. 3 and FIG. 9). Among several PDIs identified, one network showed high connectivity with few drugs interacting with 2 or 3 protein targets (FIG. 3A). We selected those PDIs where the drugs interacted with more than one protein (FIG. 3B).

To better inform potential drug selection, the direction of protein interactions in the PDI was considered (FIG. 3B). Hepatocyte Growth Factor (HGF) and Myeloperoxidase (MPO) had the top out-interactions (effector) with 12 and 11 outward interactions, respectively, followed by CXCL10 which had seven out-interactions (FIGS. 3A&B). Hence, drugs targeting HGF, MPO and CXCL10 are expected to influence most of the network's interactions. Flutamide, a nonsteroidal antiandrogen, can target ACE2 and MPO which were upregulated more than two-fold, and IL2RA which showed 1.7-fold upregulation in patients with severe disease. The immunomodulatory drug thalidomide is another example that targets four upregulated proteins: IL6R, Von Willebrand factor (VWF) and HGF with more than two-fold upregulation and F2R with 1.5-fold upregulation in severe disease.

The molecular severity score: a 46-protein signature for COVID-19 severity.

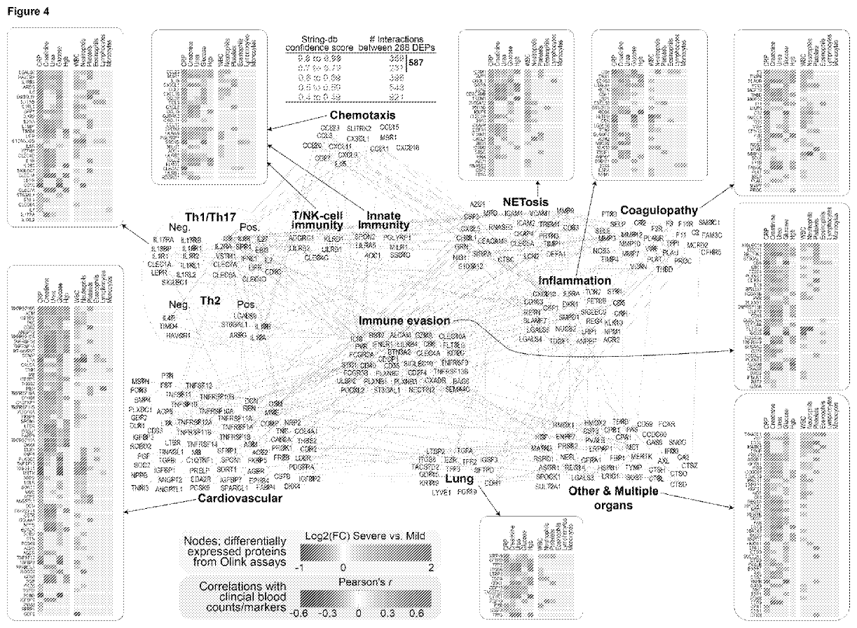

We aimed to develop a blood-based protein signature that can predict SARS-CoV-2 infected patients at higher risk of developing severe complications. The MUVR tool (2) was used for variable selection and validation in multivariate modelling to identify the most stable DEPs that can differentiate all groups (patients and control subjects), severe versus mild disease, or severe or mild disease versus controls. Four predictive models, MUVR modules, were identified through selecting the minimum number of DEPs with the least frequency of misclassifications (FIG. 4A). Module i had 10 DEPs which were common in three out of the four analyses whereas module ii had 36 DEPs that were shared in the analysis of all patients and the comparison of severe versus mild disease. Modules iii and iv consisted of DEPs unique for all patients and patients with severe versus mild disease, respectively. ROC curves found that each of the four modules had a statistically significant predictive potential (p<0.0001) with high AUC (FIG. 4B). For patients with severe disease versus patients with mild disease and controls, modules ii and iv had AUC of 1 (100% specificity and sensitivity). All modules except module iii could differentiate patients with severe disease versus controls with 100% specificity and sensitivity. Modules ii and i were the best predictors of severity (FIG. 4B), thus these two modules, consisting of 46 DEPs, were combined to calculate the “COVID-19 molecular severity score.” The molecular severity score had 100% sensitivity and specificity for all comparisons except for patients with mild disease versus controls; however, the significant predictive capacity endured (FIG. 4C).

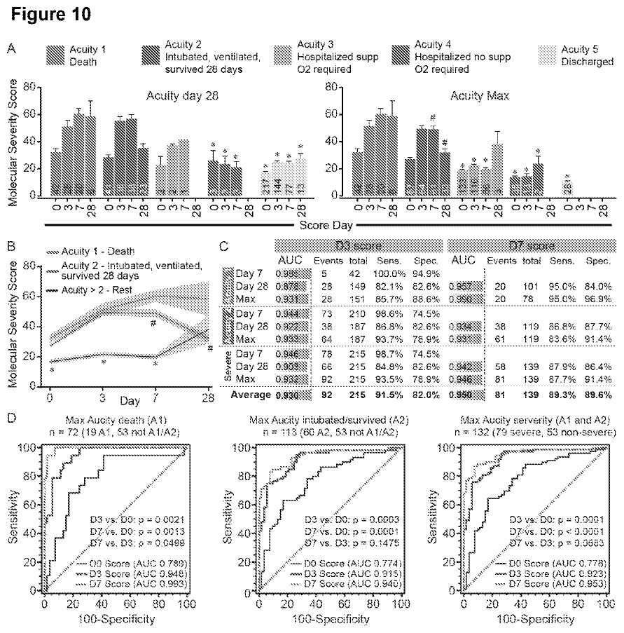

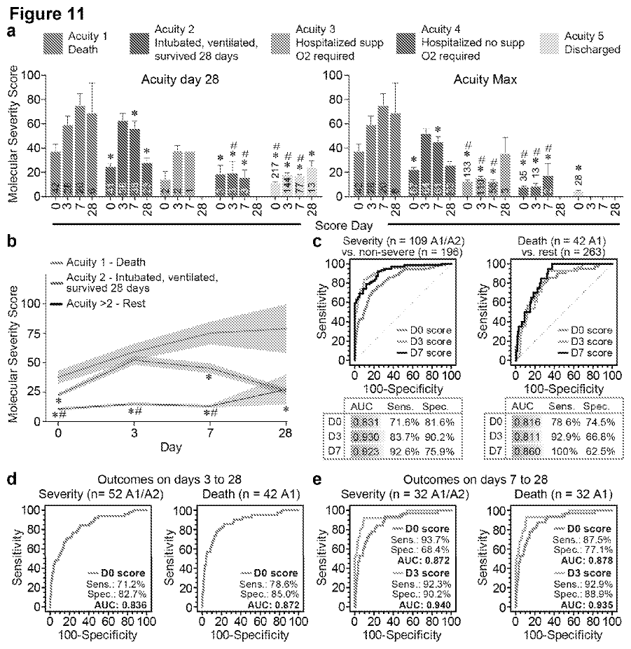

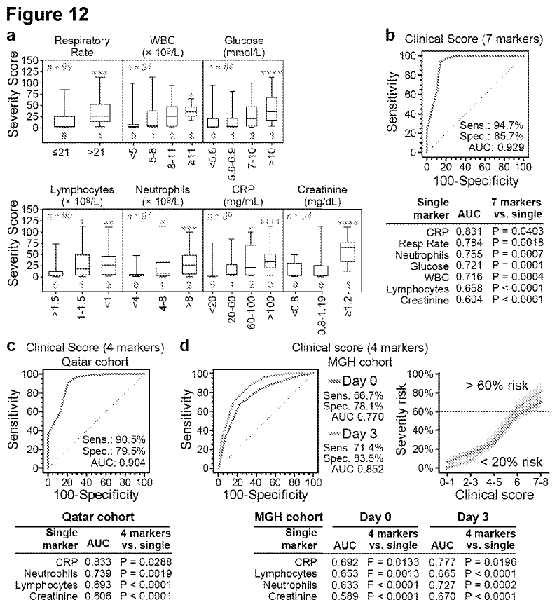

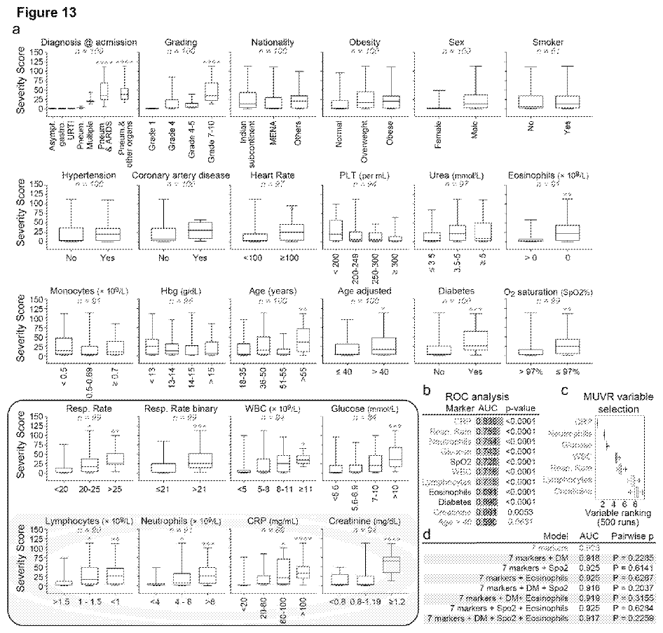

Interestingly, although variable selection in MUVR is blinded to any biological information, the selected proteins had high interactions forming a solitary connected network from 37 (80%) out of the 46 DEPs (FIG. 4D). Gene ontology biological process (GO-BP) enrichment analysis of this 46-protein signature identified the same enriched processes described earlier (FIG. 2 and FIGS. 7A-E), indicating that this minimum set of DEPs recapitulated the larger differential profiles. The complete GO-BP enrichments can be classified into five broad processes: viral entry, signaling, immune response, metabolism, and development (FIG. 10).

The COVID-19 molecular severity score validates in an independent cohort.

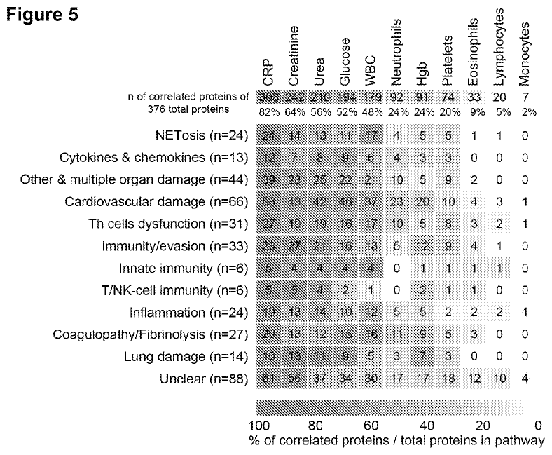

To validate the COVID-19 molecular severity score (46-protein signature), we calculated this score for patients in the independent Massachusetts General Hospital (MGH) cohort (described in Methods). As shown in FIG. 5A, this cohort consisted of three groups: group 1 included 35 patients who were assayed at all time points up to 28 days, group 2 consisted of 97 patients assayed up to 7 days, and group 3 had 72 patients assayed on days 0 and 3. The remaining patients were assayed only at a single time point and were not used in the analysis. In all patient groups, the molecular severity scores increased on day 3, and patients showed two different temporal patterns; subgroups “a” had an increasing molecular severity score, while scores for subgroups “b” reduced to baseline on days 3, 7 or 28 (FIG. 5A). None of the groups showed any significant differences in the clinical presentation, which included fever, respiratory and gastrointestinal symptoms (data not shown). However, there was a significant difference in the clinical outcome among the groups where group 1, particularly 1a, required intensive care treatment and exhibited higher mortality rate (FIG. 5B). Groups 2 and 3 had significantly fewer requirements than group 1, and the “a” subgroups (subgroups 2a and 3a) required more intensive care while the “b” subgroups (2b and 3b) had a significantly higher percentage of patients who were discharged on days 7 and 28.

Altogether, our analysis of the molecular severity scores over time showed an association with clinical outcomes. To test this directly, ROC curves were used to evaluate the molecular severity scores in the MGH cohort. Intensive care was used to define severe outcomes whereas acute care and hospital discharge (no hospitalization) were defined as mild outcomes. The molecular severity scores calculated on day 0 were significantly associated with clinical outcomes on days 3, 7, and 28 and the worst outcome within the 28 days (all outcomes), and death (FIG. 5C). However, the molecular severity scores calculated from samples collected on days 3 and 7, particularly on day 7, were better predictors of clinical outcomes and survival. This cross-validation in the MGH cohort showed that the molecular severity score could be applied on days 3 and 7 post admission to predict disease severity, expected level of care and survival up to 28 days.

A molecularly trained clinical score to predict COVID-19 severity.

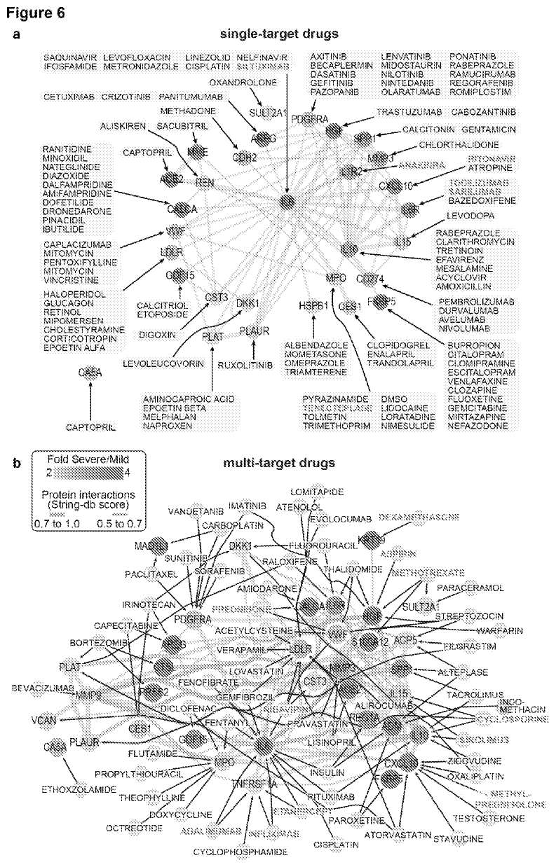

We hypothesized that the molecular severity score could be used to identify informative clinical parameters to triage SARS-CoV-2 infected patients into high or low risk for developing severe complications. A comparative analysis between the molecular severity scores and clinical parameter found that 13 out of the 24 parameters available in our cohort showed significant associations with the molecular severity score (FIG. 6S). MUVR determined that 8 of these are the most informative (FIG. 6A). These eight parameters were combined to calculate the “Clinical Risk Score”; a molecularly trained score where each clinical measure was weighted according to its molecular severity score. The Clinical Risk Score outperformed each of the single parameters (p<0.0001 for all pairwise comparisons of ROC AUCs) and showed more than 90% sensitivity and specificity to differentiate between patients with severe and mild disease (FIG. 6B). In conclusion, both the 46-protein severity score and the trained clinical score may be used during admission to identify SARS-CoV-2 infected patients who are at risk for developing severe complications. Moreover, as shown in FIG. 6C (top and middle panels), several highly up- and down-regulated proteins may be used for molecular monitoring disease progression or could serve as candidates therapeutic targets for intervention.

### DISCUSSION

Demographics of the current cohort of SARS-CoV-2 infected patients had similar characteristics as the nation-wide cohort study of the first consecutive 5,000 patients with COVID-19 in Qatar (3). While our study selected patients between 18 and 65 years of age, both cohorts consisted largely of males with a younger median age due to the relatively younger population in Qatar. Risk factors of ICU admission in our study and the national cohort study (3) included older age, male sex, higher BMI, and preexisting diabetes and hypertension. Other comorbidities such as chronic artery disease, liver disease or kidney disease were identified in both studies but did not reach significance in our smaller cohort. Of the first 5,000 consecutive cases in Qatar, 1424 patients (28.5%) required hospitalization, out of which 108 (7.6%) were admitted to ICU, and only 14 patients (0.28%) had died by 60 days after infection. In a relatively younger national cohort in Qatar, with a low comorbidity burden, COVID-19 was associated with low mortality (3), which was also reflected in our smaller cohort.

The Qatari population, with respect to COVID-19, is unique in the demographic characteristics (e.g. predominantly males with younger age) when compared to other populations such as that described in the ISARIC (1). However, our study of plasma profiling of SARS-CoV-2 infected patients and control subjects using the Olink Proteomics panels may be generalizable as the biological processes and pathways enriched in patients with severe complications in our subpopulation have also been reported in other studies. Additionally, the COVID-19 molecular severity score reported here was cross-validated in an independent, larger cohort from the Massachusetts General Hospital (MGH, USA).

Medical history of macular degeneration and of coagulation disorders (thrombocytopenia, thrombosis, and hemorrhage) were considered risk factors for higher morbidity and mortality in a recent study on 11,116 patients infected patients with SARS-CoV-2 (4). Moreover, RNA-Seq profiles from nasopharyngeal swabs in this study found several enriched immune-modulatory functions in SARS-CoV-2 infected patients versus controls, such as inflammatory response, interferon alpha response, and IL6-JAK-STAT3 signaling, which were also identified in our study. Activation of the complement and coagulation cascades was also among the most enriched gene sets (4), an observation corroborated by our plasma protein profiling results.

A multiplexed biomarker profiling of plasma from 49 SARS-CoV-2 infected patients (40 in ICU and 9 in non-ICU units) and 13 non-COVID-19, non-hospitalized controls identified multiple proteins in association with ICU admission and mortality, including HGF, RETN, LCN2, G-CSF, IL-6, IL-8, IL-6, IL-10, IL1RA and TNF-α (5), which were confirmed in our study. Importantly, the study also reported a unique neutrophil activation signature composed of neutrophil activators (G-CSF, IL-8) and effectors (RETN, LCN2 and HGF), with a strong predictive value to identify critically ill patients whereby the effector proteins strongly correlated with absolute neutrophil count (5). Our study not only identified those components of the neutrophil activation signature, but also found that the COVID-19 molecular severity score, a more comprehensive signature, also correlated with absolute neutrophil counts. Moreover, the neutrophil count was selected in the COVID-19 clinical risk score developed in our study. Another study deployed Olink Proteomics panels to measure 1,161 plasma proteins from 20 patients, 10 SARS-CoV-2 positive and 10 SARS-CoV-2 negative patients, admitted to ICU and 10 healthy controls (6). This study had a small sample size, could not determine changes contributing to ICU admission and only reported mortality. Interestingly, it uncovered similar proteins and pathways as those identified in our study in association with COVID-19 severity, such as interleukins, CXCLs/chemokines, membrane receptors linked to lymphocyte-associated microparticles, cytoplasmic/cytoskeletal proteins, and nuclear proteins or transcription factors (6). Among their reported 20 top proteins differentiating patients with COVID-19 disease from healthy controls, 13 (65%) were also confirmed in our study, with two of them were components of our COVID-19 molecular severity score, namely IL6 and IL18R1. Of their reported 20 top proteins which differentiated ICU-admitted patients with COVID-19 versus non-COVID-19 disease, 12 (60%) were also found in our study with two were components of the molecular severity score, KRT19 and CCL7.

Besides Olink technology, mass spectroscopy was used in two studies to identify deregulated proteins in SARS-CoV-2 infected patients. The first study used liquid chromatography-mass spectrometry (LC-MS) to profile 31 patients with SARS-CoV-2 infection, where the disease severity was graded according to the WHO outcome scale. The study identified 27 potential biomarkers that were differentially expressed (7). Although none of these biomarkers was identified in our study, the biological functions reported in their study were also captured in our analysis, including complement factors and the coagulation system, inflammation modulators, and pro-inflammatory factors upstream and downstream of interleukin 6. The second study used mass spectroscopy for proteomics and metabolomics analysis of sera from 46 COVID-19 and 53 control individuals and identified 93 proteins which were differentially expressed in sera from severe COVID-19 patients (8). Of these 93 differentially expressed serum proteins, 17 were included in our panel profiling of plasma with 11 (65%) of these proteins were also significantly deregulated in our cohort. More specifically, 8 of the 11 proteins were upregulated (VWF, PVR, GRN, NID1, VCAM1, SAA4, CD59, CDH1), whereas the remaining three proteins were downregulated (FETUB, APOM, and IGFBP3) in the severe cases in our study. Using mass spectroscopy, the authors developed a model of 22 proteins and seven metabolites (29 sera factors) to stratify patients according to severity (8). None of the protein biomarkers reported in this study was identified in our study; however, there is a strong overlap in biological functions, including the release of IL-6 and TNF-a, inflammatory responses, activation of the complement system and protein phosphorylation.

Interestingly, both MS-based studies agreed on ten protein biomarkers in their classifiers, 10/27 (37%) for the first study which included serum and plasma (7), and 10/22 (45%) in the second study which only used serum (8). Although our study agrees on the biological functions identified in the two MS-based studies, the lack of agreement with the named biomarkers may be due to the use of plasma in our study compared to serum in the MS studies. We cannot exclude that the MS-based studies are more comprehensive and less biased than the panel profiling used in our study. However, it should be noted that there was a small overlap between all the proteins detected by mass spectroscopy in sera (prior to statistical analysis) from the Shen et al. study (8) and the proteins profiled in plasma in our study; 134 common proteins out of the 791 (17%) proteins detected by mass spectroscopy and the 894 (15%) proteins profiled in our study.

Altogether, our study identified several biological pathways described in previous proteomic studies of sera or plasma of patients with severe COVID-19 complications. In addition to their potential biomarker value, the protein profiles can also be used to predict potential drugs for intervention. Our drug-protein interaction analyses shortlisted HGF, MPO and CXCL10 as targets that could influence most of the interactions between the plasma proteins upregulated in severe COVID-19 cases. Notable examples of possible drugs include flutamide which can target MPO, ACE2 and IL2RA and has been proposed as a possible drug for COVID-19 treatment based on ACE2 interaction network analysis (9). Methylene Blue can modulate MPO, VWF and CPA1, which were upregulated in our severe cases and had been tested in combination with other drugs in a clinical trial with critically ill COVID-19 patients in Iran (10), whilst a broader clinical trial (NCT04370288) has been designed. Thalidomide is another example that targets one of the shortlisted proteins (HGF) in addition to three other upregulated proteins in severe cases (IL6R, VWF, and F2R). Its use for COVID-19 was reported for a single case in China (11) and led to recovery, and two clinical trials (NCT04273581 and NCT04273529) have been registered. However, thalidomide's side effects and its previous dark past has been raised as serious concerns for its use to treat COVID-19 patients, and may have to be strictly limited to use in men and post-menopausal women (12).

Furthermore, methotrexate inhibits HGF and two other upregulated proteins in severe cases in our study, S100A12 and SULT2A1. This drug has been reported to inhibit SARS-CoV-2 virus replication in vitro via purine biosynthesis, thereby potently inhibiting viral RNA replication, viral protein synthesis, and virus release. As such, methotrexate was proposed as an effective measure to prevent possible COVID-19 complications (13). The use of methotrexate to treat COVID-19 patients or prevent complications has not been tested; however, a large comparative cohort study suggested that patients with recent TNF inhibitors and/or methotrexate exposure do not have increased COVID-19 related hospitalization or mortality (14).

In addition to the potential targeting of HGF, MPO and CXCL10 as highly interconnected proteins, our analysis identified ribavirin as a treatment option based on the upregulation of VWF and CST3 (Cystatin C) in patients with severe COVID-19 complications. Ribavirin, an oral nucleoside analogue, has been tested in combination with injectable interferon beta-1b and the oral protease inhibitor (lopinavir-ritonavir) in a randomized phase 2 trial to treat COVID-19 patients. Compared to lopinavir-ritonavir alone, the triple combination was safe and effectively shortened the duration of virus shedding, decreased cytokine responses, alleviated symptoms, and facilitated the discharge of patients with mild to moderate COVID-19 disease (15). A follow-up trial has been registered (NCT04494399) to test the combination of ribavirin with interferon beta-1b without lopinavir-ritonavir to treat patients with COVID-19.

In conclusion, our study identified deregulated proteins in the plasma of patients with severe COVID-19 complications that may inform therapeutic interventions. The 46-protein signature identified in our study was developed as the COVID-19 molecular severity score and used to stratify patients according to COVID-19 severity in an independent cohort. The COVID-19 molecular severity score could predict outcomes up to 28 days post-admission and from as early as three days of admission. We used the molecular severity score to select clinical parameters available at the time of admission and generated a scoring system to develop the molecularly trained clinical risk score. The molecular severity and the clinical risk scores developed here have the potential to stratify SARS-CoV-2 infected patients at early stages according to their risk of developing complications to prospectively inform healthcare management and clinical decision to prevent complications and mortality.

Methods

Patients Recruitment

A cohort of 100 patients (mild-moderate and severe) affected by COVID-19 disease and admitted to Hamad Medical Corporation (HMC) hospitals; tertiary level hospitals in Doha, Qatar, were recruited. Infection was confirmed by positive RT-PCR assays for SARS-CoV-2 from sputum and throat swab with CT values around 30. Patients with severe COVID-19 were defined as those requiring ICU admissions due to COVID19 disease or disease complications, while patients with mild-moderate COVID-19 were admitted to community hospitals but did not require ICU care. Fifty control subjects were recruited at the CRC of the Anti-Doping Laboratory Qatar from volunteers identified by Qatar Red Crescent Society, according to the criteria of being healthy, without prior history of confirmed COVID-19 infection diagnosis, normal oxygen saturation, and vital signs. Control subjects were age, sex and ethnicity matched to the patients. Individuals with poor cognitive ability, or any past or present medical disease or were not able to consent were excluded.

Samples Collection and Processing

Peripheral blood was collected within five to seven days of admission into commercially available EDTA-treated tubes, and plasma and peripheral blood mononuclear cells (PBMCs) fractions were separated using Ficoll. PBMCs were saved for use in other studies. Plasma was stored at −80° C. until further analysis.

Olink Proteomic Assays

Plasma samples were profiled in house using the proximity extension assays (PEA), 96-plex immunoassay developed by Olink Proteomics (Uppsala, Sweden) (16) following the standard protocol at Qatar Biomedical Research Institute's (QBRI) Olink certified proteomics core facility. Quality control and data normalization according to internal and external controls were carried out using the Normalized Protein eXpression (NPX) software and every run was checked and validated by the Olink support team in Uppsala. Ten different panels focused on a specific disease or biological process were used in our study; panel names are stated in the results.

Bioinformatics

For the analysis of Olink assays, the protein expression values, as log 2 of Normalized Protein eXpression (NPX), were used. Two approaches were used in the analysis; single-panel and combined-panels analyses before confirming the overlap between the two approaches. Olink data that did not pass quality control were excluded from the analyses. R packages for hierarchical clustering (heatmap.2), principal component analysis (PCA, prcomp), differentially expression analysis (Linear Models for Microarray Data (limma)), volcano plots, gene-ontology biological process (GO-PB) and KEGG pathways enrichment analyses were used through the standalone version of iDEP.92 (17) installed in RStudio (version 1.2.5).

For variable selection and validation, the algorithm for multivariate modeling with minimally biased variable selection in R (MUVR) was used in RStudio as previously described (2). MUVR is a statistical validation framework, incorporating a recursive variable selection procedure within a repeated double cross-validation (rdCV) scheme. Differentially expressed proteins selected by MUVR were used to develop protein signatures represented as meta-protein scores calculated as the ratio of average expression of NPX values of upregulated proteins to the average expression of NPX values of downregulated proteins. Upregulated and downregulated proteins were defined according to the score. For example, if the score was from the comparison of severe versus mild COVID patients, we used the upregulated or downregulated in the severe versus mild patients. Scores were evaluated using receiver operating characteristic (ROC) curve analyses to determine the area under the ROC curve (AUC), sensitivity, specificity, and significance (P<0.05) using MedCalc® (version 12.7, MedCalc Software Ltd., Belgium).

Protein-protein interaction (PPI) was analyzed and visualized using the STRING database (STRING-db version: 11.0) (18) accessed through Cytoscape (version: 3.7.2) (19). Protein-drug interaction (PDI) was analyzed using the Drug-Gene Interaction database (DGIdb, v3.0.2) (20), only using FDA-approved drugs, and interaction networks were visualized in Cytoscape.

Validation of the COVID-19 Molecular Severity Score in the MGH Cohort

To validate the COVID-19 molecular severity score (46-protein signature) developed here, we used the Massachusetts General Hospital (MGH) cohort (Data provided by the MGH Emergency Department COVID-19 Cohort (Filbin, Goldberg, Hacohen) with Olink Proteomics). The MGH cohort enrolled 384 acutely-ill patients, 18 years or older patients, with a clinical concern for COVID-

19 upon arrival in the emergency department; specifically, the patients presented with acute respiratory distress and at least one of the following: 1) tachypnea 2: 22 breaths per minute; 2) oxygen saturation:S 92% on room air; 3) a requirement for supplemental oxygen; or 4) positive-pressure ventilation. SARS-CoV-2 positivity was reported for 306 patients (80%), and 78 patients were negative. Plasma samples from positive patients (days 0, 3, 7, and 28) and negative patients (only day

0) were subjected to Olink Proteomics to measure the expression of more than 1400 proteins. We focused on the SARS-CoV-2 positive patients since the MGH cohort did not investigate the SARS-CoV-2 negative patients beyond day 0, and our interest was to determine the behavior of our COVID-19 molecular severity score over time and in relation to COVID-19 severity and outcomes. The COVID-19 molecular severity score was calculated as described above (meta-protein score) for each throughout the study. The performance of the COVID-19 molecular severity scores in the MGH cohort was evaluated with ROC curve analysis using MedCalc® (version 12.7, MedCalc Software Ltd., Belgium).

Statistics

Patient clinical data analysis was performed using Statistical Package for Social Sciences (SPSS v26, Chicago IL, USA). Groups were compared using the chi-square test, and Fisher's exact test (two-tailed) replaced the chi-square in the case of a small sample size where the expected frequency is less than 5 in any group. The results were presented as mean f SD for normally distributed data or median (IQR) for skewed results and/or number and percentage of participants as appropriate. The level of statistical significance was set at p<0.05. GraphPad Prism (version 8.4.3, GraphPad Software LLC, CA, USA) was used to compare protein signature scores across clinical subgroups using unpaired, two-tailed t-tests or one-way ANOVA with Dunnett's multiple testing correction.

It should be understood that various changes and modifications to the aspects and embodiments described herein will be apparent to those skilled in the art. Such changes and modifications can be made without departing from the spirit and scope of the present subject matter and without diminishing its intended advantages. It is therefore intended that such changes and modifications be covered by the following claims.

1. A method of treating a SARS-CoV-2 infection in a subject in need thereof, comprising administering to the subject a therapeutically effective amount of a drug, wherein the drug comprises:
A) adalimumab, alirocumab, alteplase, amiodarone, atenolol, atezolizumab, atorvastatin, bevacizumab, bortezomib, capecitabine, carboplatin, cisplatin, cyclophosphamide, cyclosporine, dexamethasone, diclofenac, didanosine, doxycycline, etanercept, ethoxzolamide, evolocumab, fenofibrate, fentanyl, filgrastim, fluorouracil, flutamide, gemfibrozil, imatinib, indomethacin, infliximab, insulin, irinotecan, lisinopril, lomitapide, lovastatin, mercaptopurine, methotrexate, methylprednisolone, octreotide, oxaliplatin, paclitaxel, paraceramol, paroxetine, pravastatin, prednisone, progesterone, propylthiouracil, raloxifene, ribavirin, rituximab, simvastatin, sirolimus, sorafenib, stavudine, streptozocin, sunitinib, tacrolimus, testosterone, thalidomide, theophylline, vandetanib, verapamil, warfarin, or zidovudine, or combinations thereof;
B) a combination of 1) ribavirin with infliximab or etanercept and with or without methylprednisolone, 2) ribavirin with methylprednisolone and with or without cyclosporine, or 3) ribavirin with sirolimus and with or without methylprednisolone; or
C) anakinra, arsenic trioxide, aspirin, atorvastatin, atropine, bevacizumab, chorionic gonadotropin, citric acid, corticotropin, crizotinib, cyclophosphamide, cyclosporine, dexamethasone, dextrose, doxycycline, eopoetin alfa, epoetin alfa, ethoxzolamide, fluorouracil, flutamide, indomethacin, lopinavir, megestrol acetate, mesalamine, methotrexate, methylene blue, methylprednisolone, octreotide, oxandrolone, paclitaxel, ribavirin, ritonavir, sirolimus, sorafenib, stabudine, stavudine, streptozotocin, sunitinib, tenecteplase, testosterone, thalidomide, theophylline, tretinoin, urokinase, verapamil, vitamin K, zoledronic acid, or zonisamide, or a combination thereof.

2. The method of claim 1, wherein the subject has been diagnosed with COVID-19.

3. The method of claim 1, wherein the drug is flutamide, lopinavir, mesalamine, methotrexate, methylene blue, ribavirin, ritonavir, or thalidomide, or a combination thereof.

4. The method of claim 1, wherein the drug is anakinra, arsenic trioxide, aspirin, atorvastatin, atropine, bevacizumab, chorionic gonadotropin, citric acid, corticotropin, crizotinib, cyclophosphamide, cyclosporine, dexamethasone, dextrose, doxycycline, eopoetin alfa, ethoxzolamide, fluorouracil, flutamide, indomethacin, megestrol acetate, methylprednisolone, octreotide, oxandrolone, paclitaxel, sirolimus, sorafenib, stabudine, streptozotocin, sunitinib, tenecteplase, testosterone, thalidomide, theophylline, tretinoin, urokinase, verapamil, vitamin K, zoledronic acid, or zonisamide, or a combination thereof.

5. The method of claim 1, wherein the drug is anakinra, bevacizumab, citric acid, crizotinib, cyclophosphamide, dexamethasone, dextrose, ethoxzolamide, indomethacin, paclitaxel, sorafenib, sunitinib, or zonisamide, or a combination thereof.

6. The method of claim 1, wherein the drug is cyclosporine, fluorouracil, sirolimus, streptozotocin, tenecteplase, or tretinoin, or a combination thereof.

7. The method of claim 1, wherein the drug is arsenic trioxide, aspirin, atorvastatin, atropine, chorionic gonadotropin, cyclosporine, doxycycline, epoetin alfa, fluorouracil, flutamide, megestrol acetate, methylprednisolone, octreotide, oxandrolone, sirolimus, stavudine, streptozotocin, tenecteplase, testosterone, thalidomide, theophylline, tretinoin, urokinase, verapamil, vitamin K, or zoledronic acid, or a combination thereof.

8. The method of claim 1, wherein the subject comprises an at least 1.5-fold upregulation, independently, of one or more expressed proteins selected from ACE2, ACP5, ADM, AREG, CA3, CA5A, CALCA, CD274, CD38, CD40, CDH2, CES1, CST3, CTSL, CX3CL1, CXCL10, DCN, DKK1, EPHB4, F3, FAS, FKBP4, FKBP5, FST, GALNT2, GDF15, HGF, HMOX1, HSPB1, IGFBP1, IL10, IL15, IL1R2, IL2RA, IL4R, IL5RA, IL6, IL6R, KLRD1, KRT19, LDLR, LEPR, MAD1L1, MERTK, MME, MMP3, MMP7, MMP9, MPO, NUCB2, PCSK9, PDGFRA, PLAT, PLAUR, PRSS2, REG1A, REN, S100A12, SMPD1, SPP1, SULT2A1, TFF3, TFPI, THBD, THBS2, TNFRSF10A, TNFRSF11A, TNFRSF11B, TNFRSF1A, TNFRSF1B, TYMP, VCAM1, VCAN, or VWF as compared to a corresponding average protein expression from a control cohort of subjects not infected with SARS-CoV-2 or as compared to a corresponding average protein expression from a cohort of SARS-CoV-2 infected subjects with no symptoms or mild-moderate symptoms.

9. The method of claim 8, wherein the at least 1.5-fold upregulation is an at least 2-fold upregulation.

10. The method of claim 1, wherein the subject comprises an at least 1.5-fold downregulation, independently, of one or more expressed proteins selected from those described in FIG. 14, panel (d) as compared to a corresponding average protein expression from a control cohort of subjects not infected with SARS-CoV-2 or as compared to a corresponding average protein expression from a cohort of SARS-CoV-2 infected subjects with no symptoms or mild-moderate symptoms.

11. The method of claim 9, wherein the at least 1.5-fold downregulation is an at least 2-fold downregulation.

12. The method of claim 1, wherein the subject comprises a molecular severity score of at least 20
wherein the molecular severity score is calculated by the average expression of the set of 10 proteins IL6, IL1RL1, SMOC1, KRT19, PTX3, TNC, AREG, HGF, TNFRSF10B, and IL18R1, divided by the average expression of the set of 2 proteins MSTN and CLEC4C.

13. The method of claim 1, wherein the subject comprises a molecular severity score of at least 15
wherein the molecular severity score is calculated by the average expression of the set of 35 proteins ACE2, ADM, ANGPTL1, AREG, CCL7, CDH2, CEACAM8, CTSL, GFRA1, HAVCR1, HGF, HS3ST3B1, IGFBP2, IL15, IL17RB, IL18R1, IL1R2, IL1RL1, IL6, IL6R, KRT19, LRIG1, MATN3, NECTIN2, PDGFRA, PRTN3, PTN, PTS, PTX3, PVR, SIGLEC10, SMOC1, TINAGL1, TNC, and TNFRSF10B, divided by the average expression of the set of 11 proteins BANK1, BMP4, CLEC4C, COMP, CRTAC1, KIT, KITLG, MSTN, NTRK2, RGMA, and SKAP1.

14. The method of claim 1, wherein the subject comprises a clinical score of at least 7,
wherein the clinical score is calculated by the sum of scores for respiratory rate, whole blood cells count, glucose concentration, lymphocyte counts, Neutrophil counts, CRP level and Creatinine level as detailed in the 7-marker nomogram according to FIG. 15.

15. The method of claim 1, wherein the subject comprises a clinical score of at least 6,
wherein the clinical score is calculated by the sum of scores for Lymphocyte counts, Neutrophil counts, CRP level and Creatinine level as detailed in the 7-marker nomogram according to FIG. 16.

16. The method of claim 1, wherein the subject has been admitted to a hospital not more than about three days prior to administering the drug.
